(19) United States
(12) Patent Application Publication
(10) Pub. No.: US 2023/0065816 A1
(43) Pub. Date: Mar. 2, 2023

(54) METHODS FOR PREVENTING AND REVERSING OPIOID ANALGESIC TOLERANCE IN SUBJECTS WITH CHRONIC PAIN

(71) Applicant: The Reasearch Foundation for Suny, Albany, NY (US)

(72) Inventors: Nai-Jiang Liu, Staten Island, NY (US); Alan Gintzler, Brooklyn, NY (US)

(21) Appl. No.: 17/797,208

(22) PCT Filed: Dec. 31, 2020
(86) PCT No.: PCT/US2020/067679

§ 371 Date: Aug. 3, 2022

Related U.S. Application Data
(60) Provisional application No. 62/971,331, filed on Feb. 7, 2020.

(51) Int. Cl. A61K 38/17; A61K 31/485; A61K 9/00; A61K 31/519; A61P 25/36

(52) U.S. Cl. CPC A61K 38/177; A61K 31/485; A61K 9/0019; A61K 31/519; A61P 25/36

(57) ABSTRACT

The current disclosure provides methods for treating chronic pain. The present methods reduce opioid analgesic/antiallodynic tolerance in a subject with chronic pain and prevent the development of opioid analgesic/antiallodynic tolerance. More specifically, the inventive methods include the administration of a caveolin-1 scaffolding domain competing polypeptide (CSD) and/or a non-receptor tyrosine kinase (c-Src) inhibitor to reduce opioid analgesic/antiallodynic tolerance as well as prevent the onset of opioid analgesic/antiallodynic tolerance in a subject with chronic pain.

### CROSS REFERENCE TO RELATED APPLICATIONS

This application claims the benefit of the filing date of U.S. Provisional Application No. 62/971,331, filed Feb. 7, 2020. The content of this earlier filed application is hereby incorporated by reference herein in its entirety.

### GOVERNMENT INTERESTS

This invention was made with government support under grant no. DA043774 awarded by The National Institutes of Health. The government has certain rights in the invention.

### INCORPORATION OF THE SEQUENCE LISTING

The present application contains a sequence listing the content of which is hereby incorporated by reference.

### FIELD OF THE INVENTION

The present disclosure relates to methods for treating chronic pain and provides methods for reducing and preventing the effects of opioid analgesic tolerance. More specifically, the current disclosure provides methods for administering certain compositions to a subject with chronic pain in order to reduce tolerance to a pain medication of the opiate family such as morphine. The current disclosure also provides methods that include administering certain compositions to a subject in order to prevent development of opioid analgesic tolerance in the subject.

### BACKGROUND

Opioid dose escalation to effectively control chronic pain remains a common strategy for severe and chronic pain management with 3-4% of adults in the United States receiving long-term opioid therapy. See Dowell D, et al. (2016) MMWR Recomm Rep 65 pp. 1-49.

Many biochemical signals have been indicated in the development of analgesic tolerance. For example, known cellular adaptations to chronic morphine include: (1) augmented AC isoform-specific synthesis (Rivera, M. and A. R. Gintzler, (1998) Mol. Brain Research, 54: p. 165-169) and phosphorylation (Chakrabarti, S., L. et al. (1998) Mol. Pharmacol., 54: p. 949-953), increasing AC stimulatory responsiveness to Gβγ and Gₛα; (2) increased phosphorylation of the Gβ subunit of G-proteins (see, e.g., Chakrabarti S., et al., (2001) Proc. Nat'l. Acad. Sci. (USA), 98: p. 4209-4214, Chakrabarti, S. and A. R. Gintzler, (2003) Mol Brain Res, 119(2): p. 144-51, and Chakrabarti, et al., (2005) Mol Brain Res, 138(1-2): p. 94-103); (3) Gₛα dephosphorylation, which increases its association with μ-Opioid receptor (MOR) (see, e.g., Chakrabarti, S., et al., (2005) Mol Brain Res, 135(1-2): p. 217-24, and Chakrabarti, S. and A. R. Gintzler, (2007) Mol Pharmacol., 72: p. 753-760); (4) augmented expression/translocation of protein kinase Cγ (PKCγ) (see, e.g., Mao, J., et al., (1995) Brain Res, 677: p. 257-267, Zeitz, K. P., et al., (2001) Pain, 94: p. 245-53, and Wang, L., et al., (1996) Brain Res., 723: p. 61-69), mitogen activated protein kinase (MAPK) [35-37], Ca2+/calmodulin-dependent protein kinase IIα (CamKIIα) (see Liang, D., et al., (2004) Neuroscience, 123(3): p. 769-75), mGluR5 (see Honda, M. et al., (2013) Neurourol Urodyn., 32(7): p. 1026-30); and (5) reciprocal changes in phosphorylation of phospholipase C (PLC) s1 and R3 (see Chakrabarti, S. and A. R. Gintzler, (2003) Proc. Nat'l. Acad. Sci. (USA), 100: p. 13686-13691), altering their relative contribution to Ca2+ signaling. These adaptations underlie the complexity and multiplicity of the underlying mechanisms that may play a role in the development of analgesic tolerance.

Despite identifying many biochemical adaptations elicited by chronic morphine, a common underlying “trigger” mechanism has not been delineated. For example, immune signaling has been linked to analgesic tolerance, hyperalgesia, addiction, dependence, and withdrawal (see Hutchinson M R., et al., (2007) Scientific World Journal 7 pp. 98-111), and has been implicated as a driving factor in a variety of chronic pain syndromes (Kosek E., et al., (2015) J Neuroimmunol 280 pp. 49-55). In addition, MOR down regulation and G protein uncoupling (see, e.g., Chakrabarti, S., et al., (1995) Mol. Brain Research, 30: p. 269-278, and Sim, L. J., et al., (1996) J. Neurosci., 16(8): p. 2684-92) are often proposed to underlie opioid analgesic tolerance. However, profound opioid analgesic tolerance can be observed in the absence of MOR downregulation (see, for example, Patel, M. B., et al., (2002) Mol Pharmacol, 62(6): p. 1464-70, Yoburn, B. C., et al., (1993) J Pharmacol Exp Ther, 265(1): p. 314-20, Shen, J., A., et al., (2000) Synapse, 38(3): p. 322-7, and Trafton, J. A. and A. I. Basbaum. (2004) Neuroscience, 125(3): p. 541-3), aggregate MOR G-protein uncoupling (see Madia, P. A., et al., (2012) Pharmacol Biochem Behav, 101(1): p. 155-65), arrestin binding, or endocytosis (see, e.g., Grecksch, G., et al. (2011) J Neurosci, 31(39): p. 13890-6), events causally associated with receptor uncoupling. Furthermore, chronic morphine enhances MOR Gs coupling and signaling via adenylyl cyclase (AC)/protein kinase A (PKA). See, e.g., Chakrabarti, S., et al., (1998) Mol. Pharmacol., 54: p. 655-662, and Gintzler, A. R. and S. Chakrabarti, (2008) J Pharmacol Exp. Ther. 325(3): p. 709-13). Even further, Chakrabarti, S., et al., (2016) J. Neurochem. 139 p. 737-747 indicates that caveolin-1 plays a role in the development of chronic morphine induced tolerance through the alteration of several signaling molecules ability to interact with caveolin-1. However, these findings only establish that the scaffolding domain in caveolin-1 is mechanistically involved. The findings, however, are deficient in establishing a causal association between a caveolin-1 scaffolding domain and behavioral analgesic tolerance, i.e., findings do not show that interrupting caveolini scaffolding has the ability to block and/or reverse opioid-induced behavioral analgesic tolerance.

Together, the art reveals administration of chronic opioid treatment involves a wide spectrum of biochemical sequelae, which have been temporally associated with the development of behavioral analgesic tolerance. However, temporal associations do not constitute causal relationships. For example, treatment of an opioid receptor blocker, e.g., naloxone, can disrupt certain biochemical sequelae and the development of analgesic tolerance. These naloxone-reversible sequelae are not necessarily, causally associated with behavioral analgesic tolerance. Instead, these findings merely show that opioid receptor activity is necessary for both the biochemical changes and analgesic tolerance to occur, but does not establish causality.

In view of the foregoing, there exists a dire need for a pharmaceutical approach to the treatment of chronic pain that mitigates the negative side-effects commonly associated with opioid drugs and, in particular, to provide a treatment which prevents and/or reduces the development of analgesic tolerance.

### SUMMARY OF THE DISCLOSURE

The present disclosure relates to methods for treating chronic pain and provides methods for reducing and preventing the effects of opioid analgesic tolerance. In embodiments, the present disclosure relates to a method for reducing opioid analgesic tolerance in a subject treated for chronic pain including: administering, to the subject, an effective amount of a caveolin-1 scaffolding domain (CSD) polypeptide, wherein the subject has chronic pain and is administered chronic opioid analgesic therapy prior to or concurrently with the administration of the CSD polypeptide, and wherein said administration of the CSD polypeptide reduces analgesic tolerance in the subject. In embodiments, the CSD polypeptide is characterized as a competing polypeptide. In embodiments, the subject may be a male or female human or non-human mammal.

In some embodiments, the present disclosure relates to a method for reducing analgesic tolerance in a subject treated for chronic pain including: administering to the subject an effective amount of a non-receptor tyrosine kinase (c-Src) inhibitor, wherein the subject has chronic pain and is administered chronic opioid analgesic therapy prior to or concurrently with the administration of a c-Src inhibitor, and wherein said administration of a c-Src inhibitor reduces analgesic tolerance in the subject.

In some embodiments, the present disclosure relates to a method for reducing opioid analgesic tolerance in a subject treated for chronic pain including: administering to the subject an effective amount of: a caveolin-1 scaffolding domain (CSD) competing polypeptide together with a c-Src inhibitor, wherein the subject has chronic pain and is administered chronic opioid analgesic therapy prior to or concurrently with the administration of the CSD polypeptide and a c-Src inhibitor, and wherein said administration of the CSD polypeptide and a c-Src inhibitor reduces opioid analgesic tolerance in the subject.

In some embodiments, the present disclosure relates to a method for preventing opioid analgesic tolerance in a subject with chronic pain including: administering to the subject an effective amount of: a caveolin-1 scaffolding domain (CSD) competing polypeptide; and a c-Src inhibitor, wherein the subject has chronic pain and has not been administered chronic opioid analgesic therapy prior to said administration of the CSD polypeptide and a c-Src inhibitor.

In some embodiments, the present disclosure relates to a method for preventing opioid analgesic tolerance in a subject with chronic pain including: administering, to the subject, an effective amount of a caveolin-1 scaffolding domain (CSD) polypeptide, wherein the subject has chronic pain and has not been administered chronic opioid analgesic therapy prior to said administration of the CSD polypeptide.

In some embodiments, the present disclosure relates to a method for preventing opioid analgesic tolerance in a subject with chronic pain including: administering, to the subject, an effective amount of a c-Src inhibitor, wherein the subject has chronic pain and has not been administered chronic analgesic therapy prior to said administration of a c-Src inhibitor.

In some embodiments, the present disclosure relates to a method of treating, ameliorating, or preventing one or more symptoms of pain in a subject, including: administering a therapeutically effective amount of an opioid to a subject; and administering a therapeutically effective amount of a polypeptide having at least 90% sequence identity to SEQ ID NO: 1 or functional fragments thereof, at least 90% identity to SEQ ID NO: 2 or functional fragments thereof, a c-Src inhibitor, or a polypeptide having at least 90% sequence identity to SEQ ID NO: 1 or SEQ ID NO:2 and a c-Src inhibitor to a subject in need thereof.

In some embodiments, the present disclosure includes a method for preventing and/or reducing opioid analgesic tolerance in a subject treated for chronic pain including: administering, to the subject, an effective amount of a caveolin-1 scaffolding domain (CSD) polypeptide, wherein the subject has chronic pain and is administered chronic opioid analgesic therapy prior to or concurrently with the administration of the CSD polypeptide, and wherein said administration of the CSD polypeptide prevents or reduces analgesic tolerance in the subject. In embodiments, the subject is administered the chronic opioid analgesic therapy concurrently with the CSD polypeptide, for at least one day prior to the administration of the CSD polypeptide, for at least six days prior to the administration of the CSD polypeptide, or the subject has not been administered opioid analgesic therapy prior to the administration of the CSD polypeptide.

In some embodiments, the present disclosure includes a method for preventing and/or reducing opioid analgesic tolerance in a subject treated for chronic pain including: administering to the subject an effective amount of a non-receptor tyrosine kinase (c-Src) inhibitor, wherein the subject has chronic pain and is administered chronic opioid analgesic therapy prior to or concurrently with the administration of the c-Src inhibitor, and wherein said administration of the c-Src inhibitor prevents or reduces opioid analgesic tolerance in the subject. In embodiments, the subject is administered the chronic opioid analgesic therapy concurrently with the c-Src inhibitor, for at least one day prior to the administration of the c-Src inhibitor, for at least six days prior to the administration of the c-Src inhibitor, or the subject has not been administered opioid analgesic therapy prior to the administration of the c-Src inhibitor.

In some embodiments, the present disclosure relates to a method for preventing and/or reducing opioid analgesic tolerance in a subject treated for chronic pain including: administering to the subject an effective amount of: a caveolin-1 scaffolding domain (CSD) polypeptide and a non-receptor tyrosine kinase (c-Src) inhibitor, wherein the subject has chronic pain and is administered chronic opioid analgesic therapy prior to or concurrently with the administration of the CSD polypeptide and the c-Src inhibitor, and wherein said administration of the CSD polypeptide and the c-Src inhibitor prevents or reduces opioid analgesic tolerance in the subject. In embodiments, the subject is administered the chronic opioid analgesic therapy concurrently with the CSD polypeptide and the c-Src inhibitor, for at least one day prior to the administration of the CSD polypeptide and the c-Src inhibitor, for at least six days prior to the administration of the CSD polypeptide and the c-Src inhibitor, or the subject has not been administered opioid analgesic therapy prior to the administration of the CSD polypeptide and the c-Src inhibitor.

The illustrative aspects of the present disclosure are designed to solve the problems herein described and/or other problems not discussed.

### BRIEF DESCRIPTION OF DRAWINGS

These and other features of this disclosure will be more readily understood from the following detailed description of the various aspects of the disclosure taken in conjunction with the accompanying drawings that depict various embodiments of the disclosure, in which:

FIGS. 1A-1C are graphs that depict intrathecal (“i.t.”) CSD and PP2 block tolerance development to intrathecal morphine.

FIGS. 2A and 2B are graphs that depict intrathecal treatment with CSD and PP2 reverses intrathecal morphine tolerance once it has developed following chronic spinal morphine application.

FIGS. 3A and 3B are graphs that depict intrathecal treatment with either CSD or PP2 reverses intrathecal morphine tolerance, after tolerance has developed following the chronic subcutaneous application of systemic morphine.

FIGS. 4A, 4B and 4C are graphs that depict intrathecal CSD prolongs systemic morphine antiallodynia (von Frey test for male and female rats) and antinociception (tail flick test) and prevents antiallodynic/antinociceptive tolerance development to chronic systemic morphine in rats.

FIG. 5 is a graph that depicts intrathecal CSD and PP2 reverse spinal tolerance to systemic morphine across all doses of morphine tested, i.e., the effect is not unique to a specific dose of morphine.

FIGS. 6A and 6B are graphs that depict intrathecal CSD or PP2 reverses spinal tolerance induced by systemic morphine.

FIGS. 7A and 7B are graphs that depict, inter alia, absence of desensitization to the opioid tolerance mitigating effect of i.t. CSD.

It is noted that the drawings of the disclosure are not necessarily to scale. The drawings are intended to depict only typical aspects of the disclosure, and therefore should not be considered as limiting the scope of the disclosure.

### DETAILED DESCRIPTION OF THE DISCLOSURE

The present disclosure relates to methods for treating chronic pain and provides methods for reducing and/or preventing the effects of opioid analgesic tolerance. More specifically, the current disclosure provides methods for administering certain compositions to a subject with chronic pain in order to reduce tolerance to a pain medication. The current disclosure also provides methods that include administering certain compositions to a subject in order to prevent development of opioid analgesic tolerance in the subject.

In embodiments, the present disclosure provides a method of treating, ameliorating, or preventing one or more symptoms of pain in a subject, including: administering a therapeutically effective amount of an opioid to a subject; and administering a therapeutically effective amount of a polypeptide having at least 90% sequence identity to SEQ ID NO: 1, a non-receptor tyrosine kinase (c-Src) inhibitor, or a polypeptide having at least 90% sequence identity to SEQ ID NO: 1 and a c-Src inhibitor to a subject in need thereof. In some embodiments, administering a therapeutically effective amount of an opioid to a subject is performed prior to or concurrently with the administration of a polypeptide having at least 90% sequence identity to SEQ ID NO: 1. In some embodiments, the therapeutically effective amount of a polypeptide having at least 90% sequence identity to SEQ ID NO: 1 is an amount sufficient to reduce or prevent an analgesic tolerance in the subject.

Advantages of the present disclosure include the administration of a caveolin-1 scaffolding domain (CSD) polypeptide, which, in embodiments, may be characterized as a competing peptide, and a c-Src inhibitor, alone or in combination, to diminish or prevent chronic opioid-induced analgesic tolerance. More specifically, the present disclosure demonstrates that inhibition of Src activity (such as through administration of a c-Src inhibitor) and/or administration of a CSD attenuate analgesic tolerance in a subject being treated for chronic pain that has developed a tolerance for an opioid medication over time. Additionally, the present disclosure demonstrates that inhibition of c-Src activity (such as through administration of a Src inhibitor) and/or administration of a CSD polypeptide has a prophylactic effect for preventing the development of opioid-induced analgesic tolerance in a subject. In embodiments, the present disclosure advantageously provides a solution to the long-standing need for the treatment of chronic pain that reduces, eliminates, or prevents one or more of the undesirable side-effects associated with chronic analgesic opioid treatment (i.e., chronic opioid analgesic therapy) including opioid-induced analgesic/antiallodynic tolerance. In some embodiments, the present disclosure prevents antiallodynic and/or antinociceptive tolerance development to chronic systemic opioid administration.

In embodiments, methods of the present disclosure are premised on the discovery that inhibiting Src activity and/or caveolin-1 (Cav1) scaffolding will reduce tolerance to an opioid analgesic or prohibit the development of opioid analgesic tolerance to chronic opioid treatment. Without being limited to any one particular theory, the inventors have discovered that the administration of opioids for the treatment of chronic pain, e.g., morphine, triggers an alteration in Cav1 mediated scaffolding and Cav1 mediated signaling through the activation of Src, specifically through Src mediated phosphorylation of caveolin-1. It is shown herein that administration of a caveolin-1 scaffolding domain (CSD) polypeptide and a c-Src inhibitor, alone or in combination, diminishes chronic opioid-induced analgesic tolerance. Notably, the data provided herein demonstrate that inhibition of Src activity (such as through administration of a c-Src inhibitor) and/or administration of a CSD polypeptide attenuate analgesic tolerance in a subject being treated for chronic pain whom has developed a tolerance for opioid medication over-time. Additionally, the data herein demonstrates that inhibition of Src activity (such as through administration of a c-Src inhibitor) and/or administration of a CSD polypeptide has a prophylactic effect for preventing the development of opioid-induced analgesic tolerance in a subject.

While the present disclosure provides methods for administering certain compositions such as drugs including one or more caveolin-1 scaffolding domain (CSD) polypeptides, one or more c-Src inhibitors and/or one or more opioids as described herein, it should be understood that determination of an effective amount of the one or more disclosed compounds is within the capability of those skilled in the art, especially in light of the detailed disclosure provided herein. For example, when determining a pharmaceutically acceptable dose for administration, one of ordinary skill in the art is able to determine an amount of one or more drugs or active agents that is within the scope of sound medical judgment suitable for use in contact with the tissues of subjects without undue toxicity, irritation, allergic response, and the like, and effective for its intended use for a clinically useful period of time. In embodiments, the effective amount of a pharmaceutical composition used to affect a particular purpose as well as its toxicity, excretion, and overall tolerance may be determined in vitro, or in vivo, by pharmaceutical and toxicological procedures either known now by those skilled in the art or by any similar method yet to be disclosed.

The exact techniques used in determining an effective amount may depend on factors such as the type and physical/chemical properties of the pharmaceutical composition, the property being tested, and whether the test is to be performed in vitro or in vivo. Determination of an effective amount of disclosed compound for administration may also include determining an effective therapeutic amount and a pharmaceutically acceptable dose, including the formulation of an effective dose range for use in vivo, including in humans.

### Definitions

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

The term “administer” or “administering” are used interchangeably herein to mean the delivery of a composition such as, for example, an opioid analgesic, CSD polypeptide or c-Src inhibitor to a subject. Administration to a subject can include all suitable modes known to those of ordinary skill in the art. For example, administration as used in the present methods can include systemic or targeted modes of administration, such as injection, oral administration, topical administration, spinal injection, or intrathecal administration. In certain instances, administration means intrathecal injection to the spinal cord of a subject, intramuscular delivery, subdermal injection, orally through a pill, tablet, dissolving tab, sublingual liquid, a transdermal patch, rectally, and/or by subdermal slow release pellets.

As used herein the term “c-Src inhibitor” means a compound that inhibits c-Src and optionally one or more members of the Src family kinase member such as c-Src. In particular, the term c-Src inhibitor as referred to herein means a compound which is an inhibitor of c-Src, and is preferably a selective inhibitor of c-Src. The selectivity of any particular c-Src inhibitor for use in accordance with the present methods are well known to one of ordinary skill in the art or could be readily determined by a person skilled in the art without undue burden. By way of example only, c-Src inhibitors for use in the present methods include A/-(2-chloro-6-methylphenyl)-2-[[6-[4-(2-hydroxyethyl)-1-piperazinyl]-2-methyl-4-pyrimidinyl]amino]-5-thiazole carboxamide monohydrate, also known as dasatinib or the free-amide or an alternative pharmaceutically acceptable salt thereof, and 4-amino-5-(4-chlorophenyl)-7-dimethylethyl)pyrazolo[3,4-c pyrimidine, also known as PP2 or a pharmaceutically acceptable salt thereof.

As used herein the “degree of identity” refers to the relatedness between two amino acid sequences or between two nucleotide sequences and is described by the parameter “identity”. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety.

In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences.

As used herein the terms “drug,” “drug substance,” “active agent,” “active pharmaceutical ingredient,” and the like, refer to a compound that may be used for treating a subject in need of treatment. Non-limiting examples of such a compound include opioids, c-Src inhibitor(s), or peptide(s) such as SEQ ID NO: 1 or SEQ ID NO: 2 of the present disclosure or highly related homologues.

As used herein the terms “drug product,” “pharmaceutical dosage form,” “dosage form,” “final dosage form” and the like, refer to a pharmaceutical composition that is administered to a subject in need of treatment and generally may be in the form of tablets, capsules, sachets containing powder or granules, liquid solutions or suspensions, patches, and the like.

As used herein the term “excipient” or “adjuvant” refers to any inert substance.

“Homologue” means an entity having a certain degree of identity or “homology” with the subject amino acid sequences and the subject nucleotide sequences.

The term “indicia of opioid analgesic tolerance” or an “indicium of opioid analgesic tolerance” as used herein is a trait exhibited by a subject that is associated with or caused by chronic administration of opioid analgesic therapy. By way of example, an indicium of opioid analgesic tolerance is when a subject requires higher doses of an opioid, e.g., morphine, in order to achieve a maximum potential analgesic effect or achieve an analgesic effect that is observed when the opioid analgesic is administered to an opioid naïve (untreated) subject.

An “opioid” as used herein means any opioid analgesic composition used to treat chronic pain and that act via any of the several opioid receptors. For example, opioids for use in the methods of the present disclosure can be readily identified by one of ordinary skill in the art by determining that naloxone is able to reverse the analgesic effect thereof. A non-exhaustive list of opioids suitable for use in the present methods includes morphine, (5a,6a)-7,8-didehydro-4,5-epoxy-17-methylmorphinan-3,6-diol, and analogues and derivatives thereof, such as hydromorphine also known as dihydromorphine, 3,6-dihydroxy-(5a,6a)-4,5-epoxy-17-methylmorphinan, and diamorphine; codeine also known as 3-methylmorphine, (5a,6a)-7,8-didehydro-4,5-epoxy-3-methoxy-17-methylmorphinan-6-ol; dihydrocodeine, 4,5-a-epoxy-3-methoxy-17-methylmorphinan-6-ol; buprenorphine, (2S)-2-[(5R,6R,7R,14S)-9a-cyclopropylmethyl-4,5-epoxy-6,14-ethano-3-hydroxy-6-methoxymorphinan-7-yl]-3,3-dimethylbutan-2-ol; tramadol, 2-[(dimethylamino)methyl]-1-(3-methoxyphenyl)cyclohexanol; fentanyl also known as fentanil, A-(1-(2-phenylethyl)-4-piperidinyl)-A-phenylpropanamide; methadone, RS)-6-(dimethylamino)-4,4-diphenylheptan-3-one; oxycodone (5R,9R,13S, 14S)-4,5o epoxy-14-hydroxy-3-methoxy-17-methylmorphinan-6-one); hydrocodone (4,5a-epoxy-3-methoxy-17-methylmorphinan-6-one); meptazinol, (RS)-3-(3-ethyl-1-methylazepan-3-yl)phenol; tapentadol, 3-[(1R,2R)-3-(dimethylamino)-1-ethyl-2-methylpropyl]phenol hydrochloride; alfentanil, A/-{1-[2-(4-ethyl-5-oxo-4,5-dihydro-1H-1,2,3,4-tetrazol-1-yl)ethyl]-4-(methoxymethyl)piperdin-4-yl}-A/-phenylpropanamide; remifentanil, methyl 1-(3-methoxy-3-oxopropyl)-4-(N-phenylpropanamido)piperidine-4-carboxylate; pentazocine, (2 RS,QRS, 1 1 RS)-6, 1 1-dimethyi-3-(3-methylbut-2-en-1-yi)-1,2,3,4,5,6-hexahydro-2,6-methano-3-benzazocin-8-ol or 2-dimethylallyl-5,9-dimethyl-2′-hydroxybenzomorphan; pethidine also known as meperidine, ethyl 1-methyl-4-phenylpipehdine-4-carboxylate); dipipanone, 4,4-diphenyl-6-(1-piperidinyl)-heptan-3-one.

The term “opioid analgesic tolerance” or “opioid-induced tolerance”, as used herein means decreased efficacy of an opioid's pain-relieving effects as a function of repeated administration to a subject over time, resulting in dose escalation. In certain instances, opioid analgesic tolerance is a reduction in analgesic effect of, for example, morphine administered to a subject repeatedly in a duration of days, weeks or months requiring an increased amount of (dose) an opioid over time to obtain an equivalent level of pain-relief as exhibited with the originally prescribed/administered amount of the opioid.

As used herein the term “pharmaceutically acceptable vehicle” refers to a diluent, adjuvant, excipient or carrier with which a compound is administered.

As used herein the term “pharmaceutical composition” refers to the combination of one or more drug substances and one or more excipients such as one or more selective peptide inhibitors of the present disclosure and one or more pharmaceutically acceptable vehicles with which the one or more selective peptide inhibitors is administered to a subject.

As used herein, the term “pharmaceutically acceptable salt” refers to a salt of a compound, which possesses the desired pharmacological activity of the parent compound. Non-limiting examples of pharmaceutically acceptable salts include: acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, for example, an alkali metal ion, an alkaline earth ion, or an aluminum ion; or coordinates with an organic base such as ethanolamine, diethanolamine, triethanolamine, N-methylglucamine, and the like.

As used herein, “polypeptide” is used interchangeably with the terms “amino acid sequence”, “peptide” and/or “protein”.

As used herein, the terms “polypeptide sequence” and “amino acid sequence” are used interchangeably.

As used herein the term “prevent”, “preventing” and “prevention” of pain means (1) reducing the risk of a patient who is not experiencing symptoms of pain from developing pain, or (2) reducing the frequency of, the severity of, or a complete elimination of pain in a subject.

The term “preventing opioid tolerance” or “preventing the development of opioid tolerance” are used interchangeably to mean prohibiting the onset (development) of opioid analgesic tolerance, such as, for example, prophylactically providing a composition to a subject prior to the manifestation of opioid analgesic tolerance or an indicium thereof is detected.

“Reducing opioid analgesic tolerance” or “reduce analgesic tolerance” as used herein means to ameliorate or reverse opioid analgesic tolerance, or one or more of the biological manifestations of the disorder by interfering with one or more points in the biological cascade that leads to or is responsible for the disorder or one or more of the biological manifestations of the disorder, to alleviate one or more of the indicia or effects associated with the opioid analgesic tolerance, or to slow the progression of the disorder or one or more of the indicia of the disorder.

As used herein, the term “sequence” can either be referring to a polypeptide sequence or a nucleic acid sequence, depending on the context.

A “subject” as used herein is a subject afflicted with chronic pain or exhibits an indicium thereof. A subject with chronic pain may suffer from or have been determined to have, for example, any one of the following disorders: fibromyalgia, rheumatoid arthritis, osteoarthritis, chronic arthropathy, spinal nerve compression syndromes associated with neoplasia and/or disc herniation, chronic joint pain of any etiology associated with inflammation and/or structural joint abnormalities, post herpetic neuralgia, trigeminal neuralgia, chronic metabolic neuropathy associated with chronic pain, migraine, inflammatory pain, post-surgical pain syndromes, irritable bowel syndrome, autonomic neuropathies, and chronic pain syndrome associated with activation of central sensitization pathways. A subject can be a mammal, such as a human, rat, dog, cat, pig or goat. In a specific embodiment, the subject of the present methods is a human with chronic pain. In embodiments, the pain is characterized as a morbidity caused by opioid analgesic tolerance. In embodiments, a subject of the present disclosure may be a male or a female human, or a male or female non-human mammal.

As used herein the term “therapeutically effective amount” means the amount of a compound that, when administered to a subject for treating or preventing disease, symptom of disease, or morbidity is sufficient to have an effect on such treatment or prevention of the disease, symptom of disease, or morbidity. A “therapeutically effective amount” can vary depending, for example, on the compound, the severity of the disease, symptom of disease, or morbidity, the etiology of the disease, symptom of disease, or morbidity, comorbidities of the subject, the age of the subject to be treated and/or the weight of the subject to be treated. A “therapeutically effective amount” is an amount sufficient to alter the subjects' natural state. In embodiments, a “therapeutically effective amount” means the amount of a compound that, when administered to a subject for treating or preventing pain, or symptom of pain is sufficient to have an effect on such treatment or prevention of the pain.

As used herein the term “treat”, “treating” and “treatment” of pain means reducing the frequency of symptoms of pain, eliminating the symptoms of pain, avoiding or arresting the development of pain, ameliorating or curing an existing or undesirable symptom caused by pain, and/or reducing the severity of symptoms of pain.

### Detailed Description of Certain Embodiments

### Embodiments for Reducing Opioid Analgesic Tolerance

In embodiments, the present disclosure includes methods for reducing opioid analgesic tolerance. For example, embodiments of the present disclosure include methods for reducing opioid analgesic tolerance in a subject with chronic pain that include administering, to a subject, a caveolin-1 scaffolding domain (CSD) competing polypeptide and/or a c-Src inhibitor, such that the administration of the CSD polypeptide and/or c-Src inhibitor reduces opioid analgesic tolerance in the subject.

In embodiments, the subject of the present disclosure has chronic pain. The subject can be any mammal (male or female) that suffers from or is diagnosed with a disorder that exhibits chronic pain. In various embodiments, the subject is a human. In certain embodiments, the subject is a human diagnosed with one or more of the following chronic pain disorders: fibromyalgia, rheumatoid arthritis, osteoarthritis, chronic arthropathy, spinal nerve compression syndromes associated with neoplasia and/or disc herniation, chronic joint pain of any etiology associated with inflammation and/or structural joint abnormalities, post herpetic neuralgia, trigeminal neuralgia, chronic metabolic neuropathy associated with chronic pain, migraine, inflammatory pain, post-surgical pain syndromes, irritable bowel syndrome, autonomic neuropathies, and chronic pain syndrome associated with activation of central sensitization pathways.

In a specific embodiment, the subject is a human with neuropathic (chronic) pain, i.e., pain caused by nervous system pathology, such as peripheral nerve injury or spinal cord injury.

In embodiments, the methods of the present disclosure include the administration of a compound or molecule to a subject. In various embodiments, administration includes systemic delivery of a molecule such as an opioid in an effective therapeutic amount and/or a pharmaceutically acceptable dose. In other embodiments, administration includes local delivery of a molecule, such as by injection of for example a c-Src inhibitor in an effective therapeutic amount and/or a pharmaceutically acceptable dose, a CSD polypeptide in an effective therapeutic amount and/or a pharmaceutically acceptable dose, or an opioid in an effective therapeutic amount and/or a pharmaceutically acceptable dose.

In certain embodiments, an opioid is chronically administered to a subject systemically by, for example, oral administration, intravenously, subcutaneously, parenterally, enterally or topically. In a specific embodiment, the opioid is chronically administered to the subject systemically by subdermal implantation of, for example, morphine pellets.

In other embodiments, chronic opioid therapy is systemically administered to the subject by injection. In embodiments, an opioid is administered intrathecally such as through manual injection, a pump and or intrathecal catheter to deliver the opioid to the intrathecal space of a subject. In a specific embodiment, the opioid is administered to the subject by intrathecal injection. In a particular embodiment, the opioid is administered to the subject by transdermal patch.

In embodiments, the methods of the present disclosure include administration of a CSD polypeptide and/or c-Src inhibitor to a subject in an effective therapeutic amount and/or a pharmaceutically acceptable dose. In some embodiments, a CSD polypeptide and/or c-Src inhibitor is administered to the subject by way of non-systemic administration. In various embodiments, the CSD polypeptide and/or c-Src inhibitor is administered intrathecally such as through manual injection, a pump and or intrathecal catheter to deliver the composition to the intrathecal space of a subject. In a specific embodiment, the CSD polypeptide and/or c-Src inhibitor is administered to the subject by intrathecal injection. In embodiments, the CSD polypeptide and/or c-Src inhibitor are administered individually, or in combination, to the subject by intrathecal injection. In embodiments, the CSD polypeptide is administered by systemic administration. In embodiments, the CSD polypeptide is administered by spinal injection.

In embodiments, the c-Src inhibitor(s), opioid(s), and CSD polypeptide(s) for use in accordance with the present disclosure will normally, but not necessarily, be formulated into pharmaceutical compositions prior to administration to a subject. An opioid, CSD polypeptide or c-Src inhibitor for use in the present methods can be formulated as a pharmaceutical composition containing a particular amount of the active ingredient, e.g., an opioid, a CSD polypeptide, a c-Src inhibitor or a combination thereof. It will be appreciated by one of ordinary skill in the art that the particular formulation and carrier will at least in part depend on the specific use and type of compound. There are numerous manners of formulation known in the art, and all of those are deemed suitable for use herein (see e.g., Pharmaceutical Preformulation and Formulation: A Practical Guide from Candidate Drug Selection to Commercial Dosage Form by Mark Gibson; Informa HealthCare, ISBN: 1574911201; or Advanced Drug Formulation Design to Optimize Therapeutic Outcomes by Robert O. Williams, David R. Taft, and Jason T. McConville; Informa HealthCare; ISBN: 1420043870). The term “carrier” refers to a diluent, excipient, and/or vehicle with which an active compound is administered. The pharmaceutical compositions of the present disclosure may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may include, in addition to the carrier, any suitable binder(s), lubricant(s), suspending agent(s), coating agent(s), and/or solubilizing agent(s). In embodiments, the phrase “pharmaceutically acceptable”, as used herein, refers to salts, molecular entities and other ingredients of compositions that are generally physiologically tolerable and do not typically produce untoward reactions when administered to a mammal (e.g., human). Suitably, as used herein, the term “pharmaceutically acceptable” means approved by a regulatory agency of the Federal or a state government for use in mammals, and more particularly in humans, or listed in the U.S. Pharmacopoeia or other generally recognized texts, for example the International Union of Pure and Applied Chemistry (IUPAC) Handbook of Pharmaceutical Salts, 2011 Edition. A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

In instances where the pharmaceutical composition is administered to a subject by injection, for example, saline, dextrose or water may be used as a suitable carrier. In embodiments wherein the formulation is administered topically, via aerosol, and for intrathecal administration such formulations may be sterile aqueous solutions for topical spray or drop administration, or application as a tincture. Alternatively, suitable topical formulations include creams, ointments, foams, lotions, emulsions, etc. In a specific embodiment, where the pharmaceutical formulation is administered to the subject by intrathecal administration, the composition can be prepared as an injectable solution, suspension, or emulsion.

Furthermore, although age, gender and weight of a subject undergoing treatment for chronic pain or in accordance with the present methods does not appear to affect the specific dosages recited herein, it is contemplated that the amount of opioid, CSD polypeptide and/or c-Src inhibitor that is administered to the subject and the dosage regimen thereof could depend on one or more of a variety of factors, including the age, weight, sex and medical condition of the subject, the severity of the chronic pain disorder, the route and frequency of administration, and the particular compound employed, and thus may vary widely. For example, the opioid, CSD polypeptide and c-Src inhibitor pharmaceutical compositions thereof that is administered to the subject in accordance with the methods of the present disclosure can be administered for immediate-, delayed-, modified-, sustained-, pulsed- or controlled-release applications for example as a single or sole-therapeutic agent or may be administered as part of a combination therapy as detailed hereinafter.

In embodiments, the amount (dosage) of an opioid, CSD polypeptide and/or c-Src inhibitor of the present disclosure being administered depends on several factors, including the type and stage of the disorder, presence or absence of an auxiliary or adjuvant drug, and the subject's weight, age, health, and tolerance for the agent. Moreover, one of ordinary skill in the art would understand that the amount (dosage) of an opioid, CSD polypeptide and/or c-Src inhibitor of the present disclosure being administered may depend upon whether an opioid, CSD polypeptide or c-Src inhibitor is being administered, and that a dosage suitable for an opioid such as morphine may not be the same amount (dosage) suitable for CSD polypeptide and/or c-Src inhibitor of the present disclosure. In a non-limiting example, an opioid (such as morphine) dosage for systemic administration may be in the amount of 2 to 2,000 mg per day, a CSD polypeptide dosage may be in the amount of 0.1 to 50 μg (microgram) via non-systemic administration, and a c-Src inhibitor (such as PP2) dosage may be in the amount of 0.05 to 0.5 μg via non-systemic administration. In embodiments, morphine may be applied in 1000-20,000 μg/kg body weight per day, which equals 100-2,000 mg per day. In some embodiments, non-limiting examples of dosages of an opioid such as morphine may be, for example, about 0.2 mg per day, about 0.5 mg per day, about 1.0 mg per day, about 1.5 mg per day, about 2.0 mg per day, about 2.5 mg per day, about 3.0 mg per day, about 4.0 mg per day, about 5.0 mg per day, about 6.0 mg per day, about 7.0 mg per day, about 8.0 mg per day, about 9.0 mg per day, about 10.0 mg per day, about 12.5 mg per day, about 15.0 mg per day, about 17.5 mg per day, about 20.0 mg per day, about 25.0 mg per day, about 30.0 mg per day, about 35.0 mg per day, about 40.0 mg per day, about 50.0 mg per day, about 60.0 mg per day, about 70.0 mg per day, about 80.0 mg per day, about 90.0 mg per day, or about 100.0 mg per day, wherein the term “about” is generally understood to be within ±10%, 5%, 2%, or 1% of the indicated value. The dosage may also be within a range bounded by any two of the foregoing values. In embodiments, a non-limiting example of an opiate dosage range includes 10,000-20,000 μg/kg of body weight per day.

In some embodiments, the CSD polypeptide or c-Src inhibitor are administered in a therapeutically effective amount and may be provided in amounts described in the Example section below.

Depending on the various factors discussed above, any of the above exemplary doses of opioid, CSD polypeptide or c-Src inhibitor can be administered once, twice, or multiple times per day, week, month or year. Furthermore, routine experimentation may be used to determine the appropriate dosage regimen for each patient by monitoring the composition's effect on opioid analgesic tolerance.

In some embodiments, the chronic opioid analgesic therapy is administered either systemically (e.g., subcutaneously), or to the central nervous system (e.g., intrathecally). In some embodiments, the CSD polypeptide is administered by systemic or spinal injection.

### Embodiments for Administration of a CSD Polypeptide Alone to Reduce Opioid Analgesic Tolerance

In embodiments, a CSD polypeptide alone is administered to a subject (male or female) with chronic pain in order to reduce or reverse opioid analgesic tolerance in the subject. In embodiments, the method includes administering, to a subject, an effective amount, such as a therapeutically effective amount of a CSD competing polypeptide, such that the administration of the CSD polypeptide reduces opioid analgesic tolerance in the subject.

In embodiments, the “CSD polypeptide” refers to a cell permeable polypeptide capable of disrupting caveolin-1 signaling or scaffolding. Without wishing to be bound by the present disclosure, it is understood that CSD polypeptide may compete with caveolin-1 protein for binding to signaling molecules. In various embodiments, the CSD polypeptide administered to the subject in the present methods comprises or consists of the amino acid sequence of SEQ ID NO:1, SEQ ID NO: 2, combinations thereof, and/or functional fragments thereof. In embodiments, the CSD polypeptide used in accordance with the present methods consists of the amino acid sequence of SEQ ID NO:1. In embodiments, the CSD polypeptide is highly related to the amino acid sequence of SEQ ID NO:1 and has e.g., at least 90%, 95%, 97% or 99% sequence identity to SEQ ID NO: 1. In some embodiments, the CSD polypeptide is CALBIOCHEM® brand CSD polypeptide, and may be supplied as a trifluoroacetate salt in the form of a lyophilized solid. In specific embodiments, the CSD polypeptide used in accordance with the present methods consists of the amino acid sequence of SEQ ID NO:2. In embodiments, the CSD polypeptide is highly related to the amino acid sequence of SEQ ID NO:2 and has e.g., at least 90%, 95%, 97% or 99% sequence identity to SEQ ID NO: 2. As indicated above, the CSD polypeptide used in the present methods can be formulated as a pharmaceutical composition prior to administration to the subject. Moreover, the CSD polypeptide may be presented as a pharmaceutically acceptable salt form.

In embodiments, an effective amount of a CSD polypeptide for use in the present method includes a pharmaceutically acceptable dose. Non-limiting examples of a dose for use herein may include at least 0.2 μg CSD polypeptide, at least 0.3 μg CSD polypeptide, at least 0.4 μg CSD polypeptide, at least 0.5 μg CSD polypeptide, at least 0.6 μg CSD polypeptide, at least 0.7 μg CSD polypeptide, at least 0.8 μg CSD polypeptide, at least 0.9 μg CSD polypeptide, at least 1.0 μg CSD polypeptide, at least 2 μg, CSD polypeptide, 3 μg CSD polypeptide, at least 4 μg CSD polypeptide, at least 5 μg CSD polypeptide, at least 6 μg CSD polypeptide, at least 7 μg CSD polypeptide, at least 8 μg CSD polypeptide, at least 9 μg CSD polypeptide, at least 10 μg CSD polypeptide, at least 11 μg CSD polypeptide, at least 12 μg CSD polypeptide, at least 13 μg CSD polypeptide, at least 14 μg CSD polypeptide, 15 μg CSD polypeptide or greater. Ina specific embodiment, the amount of CSD polypeptide administered to a subject in accordance with the present method is at least 2 μg CSD polypeptide.

In various embodiments, an effective amount of a CSD polypeptide for use in the present method includes between 0.2 μg CSD polypeptide and 10 μg CSD polypeptide. In other embodiments, the amount of CSD polypeptide administered to the subject is between 0.2 μg CSD polypeptide and 8 μg CSD polypeptide, between 0.2 μg CSD polypeptide and 5 μg CSD polypeptide, between 0.2 μg CSD polypeptide and 3 μg CSD polypeptide, is between 0.2 μg CSD polypeptide and 1 μg CSD polypeptide, between 0.5 μg CSD polypeptide and 5 μg CSD polypeptide, between 0.5 μg CSD polypeptide and 3 μg CSD polypeptide, is between 0.7 μg CSD polypeptide and 5 μg CSD polypeptide, between 0.7 μg CSD polypeptide and 3 μg CSD polypeptide, is between 1 μg CSD polypeptide and 8 μg CSD polypeptide, between 1 μg CSD polypeptide and 7 μg CSD polypeptide, between 1 μg CSD polypeptide and 6 μg CSD polypeptide, between 1 μg CSD polypeptide and 5 μg CSD polypeptide, between 1 μg CSD polypeptide and 4 μg CSD polypeptide or between 1 μg CSD polypeptide and 3 μg CSD polypeptide. In specific embodiments, the amount of CSD polypeptide administered to the subject is between 2 μg CSD polypeptide and 5 μg CSD polypeptide.

In various embodiments, the amount of CSD polypeptide administered daily to a subject in accordance with the methods of the present disclosure is 0.2 μg, 0.3 μg, 0.4 μg, 0.5 μg, 0.6 μg, 0.7 μg, 0.8 μg, 0.9 μg, 1 μg, 2 μg, 3 μg, 4 μg. 5 μg. 6 μg. 7 μg, 8 μg, 9 μg, 10 μg, 11 μg, 12 μg, 13 μg, 14 μg, 15 μg, 16 μg, 17 μg, 18 μg, 19 μg 20 μg, or greater. In a specific embodiment, the amount of CSD polypeptide administered to a subject in accordance with the inventive methods is 2.0 μg.

In some embodiments, particularly those where a CSD polypeptide is administered to a subject systemically, the above amounts of CSD polypeptide may increase by about 100× depending on several factors known to those of ordinary skill in the art, such as formulation of the CSD polypeptide, the subject's weight, age, health, and tolerance for the agent. For example, in instances whereby a subject is to be provided 1-2 μg CSD polypeptide by intrathecal injection, the subject would be administered 100-200 μg CSD polypeptide systemically.

In some embodiments, the CSD polypeptide or functional fragments thereof are administered in a therapeutically effective amount and may be provided in amounts described in the Example section below.

In various embodiments, the method for reducing opioid analgesic tolerance includes administration of the CSD polypeptide after administration of an opioid analgesic to the subject. In certain embodiments, the subject is administered the CSD polypeptide at least one day after administration of an opioid analgesic therapy to the subject. In other embodiments, the subject is administered the CSD polypeptide after the subject has been administered opioid analgesic therapy for at least two days or at least three days. In certain embodiments, the subject is administered the CSD polypeptide after the subject has been administered opioid analgesic therapy for at least four days, at least five days, at least six, at least seven, at least 8, at least 9 or at least 10 days. In a specific embodiment, the subject is administered the CSD polypeptide at least 6 days after the subject has been administered an opioid analgesic therapy for the treatment of chronic pain. In another embodiment, the subject is administered the CSD polypeptide at least 7 days after the subject has been administered an opioid analgesic therapy for the treatment of chronic pain. In one embodiment, the subject is administered the CSD polypeptide after the subject has been administered the opioid analgesic therapy for at least 10 days.

In some embodiments, the method for reducing opioid analgesic tolerance reduces or eliminates one or more indicia of opioid-induced analgesic tolerance.

Therefore, in certain embodiments, the subject is determined to have one or more indicia of opioid-induced analgesic tolerance prior to administration of the CSD polypeptide. In various embodiments, the subject is administered the CSD polypeptide after the detection of hyperalgesia, which is temporally associated with opioid analgesic tolerance. In a specific embodiment, the subject is determined to require dose escalation of opioid analgesic therapy to achieve pain relief.

In some instances, the method includes administration of the CSD polypeptide concurrently with administration of opioid analgesic therapy to the subject. In some embodiments, the opioid is morphine, fentanyl, hydrocodone, hydromorphone, meperidine, oxycontin, oxycodone, or a combination thereof. In a specific embodiment, the opioid is morphine, hydrocodone, oxycontin, oxycodone or a combination thereof. In one embodiment, the opioid is morphine. In embodiments, pharmaceutically acceptable salt forms of the opioids may be included in the methods of the present disclosure.

Therefore, in various instances, the methods of the present disclosure include administering, to a subject an effective amount of a CSD polypeptide as described above and an amount of an opioid that provides pain relief to the subject.

Non-limiting examples of a therapeutically effective amount of opioid for use in the present disclosure via intrathecal administration includes at least 0.5 μg, at least 1 μg opioid, at least 1.5 μg, at least 2 μg, at least 3 μg, at least 4 μg, at least 5 μg, at least 6 μg, at least 7 μg, at least 8 μg, at least 9 μg, at least 10 μg, at least 11 μg, at least 12 μg, at least 13 μg, at least 14 μg, at least 15 μg, at least 16 μg, at least 17 μg, at least 18 μg, at least 19 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, at least 120 μg, at least 125 μg, at least 130 μg, at least 135 μg, at least 140 μg, at least 145 μg, at least 150 μg, at least 155 μg, at least 160 μg, at least 165 μg, at least 170 μg, at least 175 μg, at least 180 μg, at least 185 μg, at least 190 μg, at least 195 μg, at least 200 μg, at least 205 μg, at least 210 μg, at least 215 μg, at least 220 μg, or greater of an opioid or combination thereof.

In a specific embodiment, the amount of opioid for use in the present method includes at least 5 μg. In one embodiment, the opioid administered to the subject in accordance with the present method is morphine and the amount administered is at least 15 μg.

In various embodiments, an effective amount of an opioid for use in the present method includes between 0.5 μg opioid and 120 μg opioid. In other embodiments, the amount of opioid administered to the subject is between 1.0 μg opioid and 100 μg opioid, between 1 μg opioid and 80 μg opioid, between 1 μg opioid and 70 μg opioid, between 1 μg opioid and 60 μg opioid, between 1 μg opioid and 50 μg opioid, between 1 μg opioid and 40 μg opioid, between 1 μg opioid and 30 μg opioid, or between 1 μg opioid and 20 μg opioid. In certain embodiments, the amount of opioid for administration in accordance with the present method is between 2 μg opioid and 50 μg opioid, between 2 μg opioid and 20 μg opioid, between 3 μg opioid and 50 μg opioid, between 3 μg opioid and 20 μg opioid, between 4 μg opioid and 50 μg opioid, between 4 μg opioid and 20 μg opioid, between 5 μg opioid and 50 μg opioid, between 5 μg opioid and 20 μg opioid, between 6 μg opioid and 50 μg opioid, between 6 μg opioid and 20 μg opioid, between 7 μg opioid and 50 μg opioid, between 7 μg opioid and 20 μg opioid, between 8 μg opioid and 50 μg opioid, between 8 μg opioid and 20 μg opioid, between 9 μg opioid and 50 μg opioid, between 9 μg opioid and 20 μg opioid. In specific embodiments, the amount of opioid administered to a subject in accordance with the inventive methods is between 1.0 μg opioid and 10 μg opioid, between 2 μg opioid and 9 μg opioid, between 3 μg opioid and 8 μg opioid, between 4 μg opioid and 7 μg opioid, or between 5 μg opioid and 6 μg opioid.

In a specific embodiment, the amount of opioid administered to a subject in accordance with the inventive methods is 3 μg. In one embodiment, the opioid administered to the subject in accordance with the present method is morphine and the amount administered intrathecally is 3 μg.

In embodiments, opioid may be administered in a pharmaceutically acceptable dose suitable for treating humans. One of ordinary skill in the art may select a dosage in an effective amount to manage pain as is known in the art. In embodiments, one of ordinary skill in the art may select any opioid dosing honed over the past 50 years, and thus includes the entire dosing range developed for humans.

In a non-limiting example, the amount of opioid administered to a subject in accordance with the methods of the present disclosure is 75 mg. In another non-limiting example, the opioid administered to the subject in accordance with the present method is morphine and the amount administered systemically is 75 mg per morphine-based pellet.

In some embodiments, particularly those where an opioid is administered to a subject systemically, a systemic dose may be increased by at least about 100× of an intrathecal dose. As a non-limiting example, in instances wherein a subject is to be provided 2-3 μg of morphine by intrathecal injection, the subject may be administered 2-2,000 mg of morphine systemically per day. In certain embodiments, the opioid analgesic therapy is administered to the subject systemically or intrathecally. In some embodiments, the opioid is administered to the subject systemically, such as by oral administration, topical administration or intravenous injection. In certain embodiments, chronic opioid therapy is administered to the subject by intramuscular delivery, subdermal injection, orally through a pill, tablet, dissolving tab, sublingual liquid, a transdermal patch, rectally, and/or by subdermal slow release pellets.

In various embodiments, an opioid is administered intrathecally such as through manual injection, a pump and or intrathecal catheter to deliver the opioid to the intrathecal space of a subject. In a specific embodiment, the opioid is administered to the subject by spinal injection, or intrathecal injection such as by injection to the spinal cord of the subject.

In some embodiments, the method includes administration of the CSD polypeptide systemically or intrathecally. In certain instances, the CSD polypeptide is administered by injection. In one embodiment, the CSD polypeptide is administered to the subject intrathecally, such as by injection to the spinal cord of the subject.

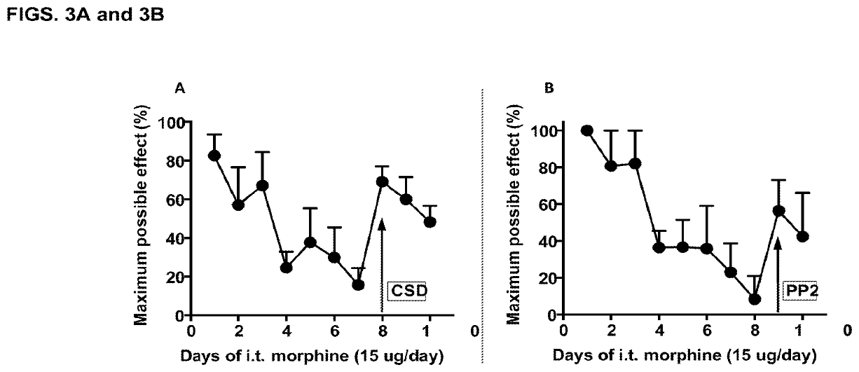

In a particular embodiment, the subject is administered chronic opioid therapy and CSD polypeptide by e.g., spinal injection into the central nervous system or intrathecally in accordance with the present methods. See for example the example section below and FIG. 3A.

In another embodiment, the inventive method includes administering the subject chronic opioid therapy systemically and administering the subject a CSD polypeptide by spinal injection or intrathecally. In a specific embodiment, the subject is administered chronic opioid therapy by implantation of subdermal slow release pellets and is administered CSD polypeptide intrathecally.

In embodiments, the present disclosure includes a method for reducing opioid analgesic tolerance in a subject treated for chronic pain including: administering, to the subject, an effective amount of a caveolin-1 scaffolding domain (CSD) competing polypeptide, wherein the subject has chronic pain and is administered chronic opioid analgesic therapy prior to or concurrently with the administration of the CSD polypeptide, and wherein said administration of the CSD polypeptide reduces analgesic tolerance in the subject. In embodiments, the subject is administered the chronic opioid analgesic therapy concurrently with the CSD polypeptide, for at least one day prior to the administration of the CSD polypeptide, or for at least six days prior to the administration of the CSD polypeptide. In embodiments, the analgesic is an opioid selected from the group consisting of morphine, fentanyl, hydrocodone, hydromorphone, meperidine, oxycontin, oxycodone, and combinations thereof. In embodiments, the chronic analgesic therapy is administered systemically, intrathecally, or by subcutaneous injection. In embodiments, the CSD polypeptide is administered by spinal injection or intrathecal injection. In embodiments, the CSD polypeptide includes the amino acid sequence consisting of SEQ ID NO:1, or an amino acid sequence having at least 90%, 95%, 97% or 99% sequence identity to SEQ ID NO:1. In embodiments, the CSD polypeptide may include variants of SEQ ID NO: 1 including 1 or more (several) conservative substitutions. In embodiments, CSD polypeptide includes functional fragments of the amino acid sequence of SEQ ID NO: 1, wherein the fragment is capable of competing with Cav1 for binding to Cav1's binding proteins, interrupting Cav1 functionality. In embodiments, the CSD polypeptide includes the amino acid sequence consisting of SEQ ID NO:2, or an amino acid sequence having at least 90%, 95%, 97% or 99% sequence identity to SEQ ID NO:2. In embodiments, the CSD polypeptide may include variants of SEQ ID NO: 2 including 1 or more (several) conservative substitutions. In embodiments, CSD polypeptide includes functional fragments of the amino acid sequence of SEQ ID NO: 2, wherein the fragment is capable of competing with Cav1 for binding to Cav1's binding proteins, interrupting Cav1 functionality.

In some embodiments, the chronic opioid analgesic therapy is administered either systemically (e.g., subcutaneously), or to the central nervous system (e.g., intrathecally). In some embodiments, the CSD polypeptide is administered by systemic or spinal injection.

### Embodiments for Administration of a C-Src Inhibitor to Reduce Opioid Analgesic Tolerance

In some embodiments, the present disclosure includes administering a c-Src inhibitor to reduce opioid analgesic tolerance. For example, in embodiments, the present disclosure includes a method for reducing opioid analgesic tolerance in a subject with chronic pain that includes administering, to a subject, a c-Src inhibitor, such that the administration of the c-Src inhibitor reduces opioid analgesic tolerance in the subject. In embodiments, an effective amount of a c-Src inhibitor alone is administered to the subject with chronic pain in order to reduce opioid analgesic tolerance in the subject.

In embodiments, any c-Src inhibitor can be used in accordance with the present methods. For example, c-Src inhibitors for use in the present methods include, but are not limited to, A/-(2-chloro-6-methylphenyl)-2-[[6-[4-(2-hydroxyethyl)-1-piperazinyl]-2-methyl-4-pyrimidinyl]amino]-5-thiazole carboxamide monohydrate (dasatinib) or the free-amide or a pharmaceutically acceptable salt thereof, and 4-amino-5-(4-chlorophenyl)-7-(dimethylethyl)pyrazolo[3,4-d] pyrimidine (PP2). In a specific embodiment, the c-Src inhibitor administered to the subject in accordance with the present methods is PP2. As described above, a suitable c-Src inhibitor for use in the present methods can be formulated as a pharmaceutical composition prior to administration to the subject.

Non-limiting examples of an effective amount of a c-Src inhibitor for use in the present method, such as a therapeutically effective amount, includes at least 0.5 μg, at least 0.7 μg, at least 0.9 μg, at least 1.0 μg, at least 1.5 μg, at least 2.0 μg, at least 2.5 μg, at least 5 μg or more of a c-Src inhibitor. In a specific embodiment, the amount of c-Src inhibitor for use in the present method includes at least 0.1 μg c-Src inhibitor. In one embodiment, the amount of c-Src inhibitor administered in accordance with the present method is at least 0.3 μg.

In various embodiments, an effective amount of a c-Src inhibitor for use in the present method includes between 0.03 μg c-Src inhibitor and 0.5 μg c-Src inhibitor. In other embodiments, the amount of c-Src inhibitor administered to the subject is between 0.03 μg and 0.4 μg, between 0.03 μg and 0.35 μg, between 0.03 μg and 0.3 μg, between 0.03 μg and 0.25 μg, between 0.03 μg and 0.2 μg, between 0.03 μg and 0.15 μg, between 0.03 μg and 0.1 μg, between 0.03 μg and 0.08 μg, between 0.03 μg and 0.06 μg, or between 0.03 μg and 0.04 μg of c-Src inhibitor. In certain embodiments, the amount of c-Src inhibitor administered to the subject is between 0.05 μg and 0.4 μg, between 0.1 μg and 0.3 μg, or between 0.1 μg and 0.4 μg, between 0.15 μg and 0.35 μg or between 0.2 μg and 0.4 μg of c-Src inhibitor.

In certain embodiments, the amount of c-Src inhibitor administered to a subject in accordance with the inventive methods is 0.01 μg, 0.02 μg, 0.03 μg, 0.04 μg, 0.05 μg, 0.06 μg, 0.07 μg, 0.08 μg, 0.09 μg, 0.1 μg, 0.2 μg, 0.3 μg, 0.4 μg, 0.5 μg, 0.6 μg, 0.7 μg, 0.8 μg, 0.9 μg, 1.0 μg, 1.1 μg, 1.2 μg, 1.3 μg, 1.4 μg, 1.5 μg, 1.6 μg, 1.7 μg, 1.8 μg, 1.9 μg, 2.0 μg, 2.1 μg, 2.2 μg, 2.3 μg, 2.4 μg, 2.5 μg, 2.6 μg, 2.7 μg, 2.8 μg, 2.9 μg, 3.0 μg, 3.1 μg, 3.2 μg, 3.3 μg, 3.4 μg, 3.5 μg, 3.6 μg, 3.7 μg, 3.8 μg, 3.9 μg, 4.0 μg, 4.1 μg, 4.2 μg, 4.3 μg, 4.4 μg, 4.5 μg, 4.6 μg, 4.7 μg, 4.8 μg, 4.9 μg or 5 μg. In a specific embodiment, the amount of c-Src inhibitor administered to a subject in accordance with the inventive methods is 0.3 μg.

In a particular embodiment, amount of the c-Src inhibitor for use in the present method is at least 0.01 μg. In one embodiment, the c-Src inhibitor administered to the subject in accordance with the present method is PP2 and the amount administered is at least 0.01 μg.

In some embodiments, particularly those where c-Src inhibitor is administered to a subject systemically, the above amounts of c-Src inhibitor may increase by at least about 100× depending on several factors known to those of ordinary skill in the art, such as the type of c-Src inhibitor administered, formulation of the inhibitor, the subject's weight, age, health, and tolerance for the agent. For example, in instances wherein a subject is to be provided 0.1-1 μg c-Src inhibitor by intrathecal injection, the subject would be administered 10 μg-100 μg of the c-Src inhibitor systemically.

In one embodiment, the method for reducing opioid analgesic tolerance includes administration of the c-Src inhibitor after administration of opioid analgesic therapy to the subject. In certain embodiments, the subject is administered the c-Src inhibitor at least one day after initiation of administration of an opioid analgesic therapy to the subject. In other embodiments, the subject is administered the c-Src inhibitor after the subject has been administered opioid analgesic therapy for at least three days, at least four days, or at least five days. In certain embodiments, the subject is administered the c-Src inhibitor after the subject has been administered opioid analgesic therapy for at least six, at least seven, at least 8, at least 9 or at least 10 days. In one embodiment, the subject is administered the c-Src inhibitor after the subject has been administered the opioid analgesic therapy for at least 6 days. In another embodiment, the subject is administered the c-Src inhibitor at least 7 days after the subject has been administered an opioid analgesic therapy for the treatment of chronic pain. In one embodiment, the subject is administered the c-Src inhibitor after the subject has been administered the opioid analgesic therapy for at least 10 days.

In some embodiments, the method for reducing opioid analgesic tolerance reduces or eliminates one or more indicia of opioid-induced analgesic tolerance.

Therefore, in certain embodiments, the subject is determined to have one or more indicia of opioid-induced analgesic tolerance prior to administration of the c-Src inhibitor. In various embodiments, the subject is administered the c-Src inhibitor after the detection of hyperalgesia. In a specific embodiment, the subject is determined to require dose escalation of opioid analgesic therapy to achieve pain relief.

In some instances, the method includes administration of the c-Src inhibitor concurrently with initial administration of opioid analgesic therapy to the subject.

Therefore, in certain instances, the methods of the present disclosure include administering, to a subject an effective amount of a c-Src inhibitor as described above and an amount of an opioid that provides pain relief to the subject, as described above.

In some embodiments, the opioid is morphine, fentanyl, hydrocodone, hydromorphone, meperidine, oxycontin, oxycodone, or a combination thereof. In a specific embodiment, the opioid is morphine, hydrocodone, oxycontin, oxycodone or a combination thereof. In one embodiment, the opioid is morphine.

In embodiments, a therapeutically effective amount of opioid for use in the present disclosure via intrathecal administration includes at least 0.5 μg, at least 1 μg opioid, at least 1.5 μg, at least 2 μg, at least 3 μg, at least 4 μg, at least 5 μg, at least 6 μg, at least 7 μg, at least 8 μg, at least 9 μg, at least 10 μg, at least 11 μg, at least 12 μg, at least 13 μg, at least 14 μg, at least 15 μg, at least 16 μg, at least 17 μg, at least 18 μg, at least 19 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, at least 120 μg, at least 125 μg, at least 130 μg, at least 135 μg, at least 140 μg, at least 145 μg, at least 150 μg, at least 155 μg, at least 160 μg, at least 165 μg, at least 170 μg, at least 175 μg, at least 180 μg, at least 185 μg, at least 190 μg, at least 195 μg, at least 200 μg, at least 205 μg, at least 210 μg, at least 215 μg, at least 220 μg, or greater of an opioid or combination thereof. In embodiments where the subject is human, greater than 220 μg may be a therapeutically effective amount.

In a specific embodiment, a therapeutically effective amount of opioid for use in the present disclosure via intrathecal administration includes at least 5 μg. In one embodiment, the opioid administered to the subject in accordance with the present method is morphine and the amount administered is at least 15 μg via intrathecal administration.

In various embodiments, a therapeutically effective amount of an opioid for use in the present method includes between 0.5 μg opioid and 120 μg opioid. In other embodiments, the amount of opioid administered to the subject is between 1.0 μg opioid and 100 μg opioid, between 1 μg opioid and 80 μg opioid, between 1 μg opioid and 70 μg opioid, between 1 μg opioid and 60 μg opioid, between 1 μg opioid and 50 μg opioid, between 1 μg opioid and 40 μg opioid, between 1 μg opioid and 30 μg opioid, or between 1 μg opioid and 20 μg opioid. In certain embodiments, the amount of opioid for administration in accordance with the present method is between 2 μg opioid and 50 μg opioid, between 2 μg opioid and 20 μg opioid, between 3 μg opioid and 50 μg opioid, between 3 μg opioid and 20 μg opioid, between 4 μg opioid and 50 μg opioid, between 4 μg opioid and 20 μg opioid, between 5 μg opioid and 50 μg opioid, between 5 μg opioid and 20 μg opioid, between 6 μg opioid and 50 μg opioid, between 6 μg opioid and 20 μg opioid, between 7 μg opioid and 50 μg opioid, between 7 μg opioid and 20 μg opioid, between 8 μg opioid and 50 μg opioid, between 8 μg opioid and 20 μg opioid, between 9 μg opioid and 50 μg opioid, between 9 μg opioid and 20 μg opioid. In specific embodiments, the amount of opioid administered to a subject in accordance with the inventive methods is between 1.0 μg opioid and 10 μg opioid, between 2 μg opioid and 9 μg opioid, between 3 μg opioid and 8 μg opioid, between 4 μg opioid and 7 μg opioid, or between 5 μg opioid and 6 μg opioid.

In a specific embodiment, the amount of opioid administered to a subject in accordance with the inventive methods is 3 μg. In one embodiment, the opioid administered to the subject in accordance with the present method is morphine and the amount administered intrathecally is 3 μg.

In a specific embodiment, the amount of opioid administered to a subject in accordance with the inventive methods is 75 mg morphine-based per pellet. In one embodiment, the opioid administered to the subject in accordance with the present method is morphine and the amount administered systemically is 75 mg morphine-based per pellet.

In some embodiments, particularly those where an opioid is administered to a subject systemically, the above amounts may be increased by about 100× depending on several factors known to those of ordinary skill in the art, such as formulation of the opioid, type of opioid, the subject's weight, age, health, and tolerance for the agent. For example, in instances whereby a subject is to be provided 2-3 μg of morphine by intrathecal injection, the subject would be administered 200-300 μg morphine systemically. In some embodiments, opioid is administered to a subject systemically in a dose of 1000-20,000 ug/kg body weight per day, which equals 100-2,000 mg per day.

The opioid therapy and c-Src inhibitor can be formulated and administered as described above. In some embodiments, the method includes administration of the c-Src inhibitor systemically or intrathecally. In certain instances, the c-Src inhibitor is administered by injection. In one embodiment, the c-Src inhibitor is administered to the subject intrathecally, such as by injection to the spinal cord of the subject.

In a particular embodiment, the subject is administered chronic opioid therapy and c-Src inhibitor intrathecally in accordance with the present methods. In a specific embodiment, the subject is administered chronic morphine by intrathecal injection to the spinal cord and is also administered PP2, a c-Src inhibitor, intrathecally.

In another embodiment, the methods of the present disclosure include administering the subject chronic opioid therapy systemically and administering the subject a c-Src inhibitor intrathecally. In a specific embodiment, the subject is administered chronic morphine by implantation of subdermal slow release pellets and is administered the PP2 (c-Src inhibitor) intrathecally.

In embodiments, the present disclosure includes a method for reducing opioid analgesic tolerance in a subject treated for chronic pain including: administering to the subject an effective amount of a c-Src inhibitor, wherein the subject has chronic pain and is administered chronic opioid analgesic therapy prior to or concurrently with the administration of the c-Src inhibitor, and wherein said administration of the c-Src inhibitor reduces analgesic tolerance in the subject. In embodiments, the subject is administered the chronic opioid analgesic therapy concurrently with the c-Src inhibitor, for at least one day prior to the administration of the c-Src inhibitor, or for at least six days prior to the administration of the c-Src inhibitor. In embodiments, the analgesic is an opioid selected from the group consisting of morphine, fentanyl, hydrocodone, hydromorphone, meperidine, oxycontin, oxycodone, and combinations thereof. In embodiments, the chronic opioid analgesic therapy is administered systemically, intrathecally, by subcutaneous injection, or by intrathecal injection. In embodiments, the c-Src inhibitor comprises 4-Amino-5-(4-chlorophenyl)-7-(dimethylethyl)pyrazolo[3,4-d]pyrimidine (PP2).

### Embodiments for Co-Administration of a CSD Polypeptide and C-Src Inhibitor to Reduce Opioid Analgesic Tolerance

In embodiments, the present disclosure includes co-administration of a CSD polypeptide and c-Src inhibitor to reduce opioid analgesic tolerance. In embodiments, a method for reducing opioid analgesic tolerance in a subject with chronic pain is provided that includes administering, to a subject, a CSD polypeptide and a c-Src inhibitor, such that the administration of the CSD polypeptide and c-Src inhibitor reduces opioid analgesic tolerance in the subject.

In embodiments, the method for reducing opioid analgesic tolerance includes administration of a CSD polypeptide and a c-Src inhibitor after initial administration of an opioid analgesic therapy has been provided to the subject. In certain embodiments, the subject is administered the CSD polypeptide and the c-Src inhibitor at least one day after administration of an opioid analgesic therapy to the subject. In other embodiments, the subject is administered the CSD polypeptide and c-Src inhibitor after the subject has been administered opioid analgesic therapy for at least three days. In certain embodiments, the subject is administered the CSD polypeptide and c-Src inhibitor after the subject has been administered opioid analgesic therapy for at least six days, at least seven days, at least eight days, at least nine days, at least ten days, at least eleven days, at least twelve days, at least thirteen days at least fourteen days, or more. In one embodiment, the subject is administered the CSD polypeptide and c-Src inhibitor after the subject has been administered chronic opioid analgesic therapy for at least 10 days.

In some embodiments, the method for reducing opioid analgesic tolerance reduces or eliminates one or more indicia of opioid-induced analgesic tolerance.

Therefore, in certain embodiments, one or more indicia of opioid-induced analgesic tolerance are detected in the subject prior to administration of a CSD polypeptide and a c-Src inhibitor. In various embodiments, the subject is administered the CSD polypeptide and c-Src inhibitor after the detection of hyperalgesia. In a specific embodiment, the subject is determined to require dose escalation of opioid analgesic therapy to achieve pain relief.

In some instances, the method includes administration of a CSD polypeptide and a c-Src inhibitor concurrently with administration of opioid analgesic therapy to the subject.

The CSD polypeptide, c-Src inhibitor and opioid analgesic therapy can be administered in accordance with the present method for reducing analgesic tolerance in any amount and route of administration described above, the entire contents of which are incorporated herein by reference.

As described above, the opioid analgesic therapy can be administered to the subject systemically or intrathecally. In some embodiments, the opioid analgesic therapy is administered to the subject systemically, such as by oral administration, topical administration, subcutaneous implantation or by intravenous injection. In a specific embodiment, the opioid is morphine, and the opioid analgesic therapy is administered to the subject by subcutaneous release of morphine pellets.

In various embodiments, the opioid analgesic therapy is administered to the subject intrathecally, such as by injection to the spinal cord of the subject. In a specific embodiment, the opioid is morphine, and the opioid analgesic therapy is administered to the subject by intrathecal injection,

In some embodiments, the method includes administration of the CSD polypeptide and c-Src inhibitor systemically or intrathecally. In certain instances, the CSD polypeptide and c-Src inhibitor are administered by injection.

In a specific embodiment, the CSD polypeptide and c-Src inhibitor are administered to the subject by spinal injection or intrathecally.

The CSD polypeptide and c-Src inhibitor can be administered together or separately through the same mode of administration or separate modes of administration. In various embodiments, the CSD polypeptide and c-Src inhibitor are formulated for co-administration in a single or separate injection, topical formulation or hard or soft gelatin capsule, pill, tablet, including a coated tablet, elixir, suspensions, syrup or inhalable. In certain embodiments, the CSD polypeptide and c-Src inhibitor are formulated for co-administration in a single dose. In one embodiment, the c-Src inhibitor and CSD polypeptide are administered to the subject intrathecally, as a single injection to the spinal cord of the subject.

In other instances, the CSD polypeptide can be administered by injection and the c-Src inhibitor can be administered to the subject subcutaneously, or vice versa. In some instances, administration includes administering, to the subject, a pharmaceutical formulation that includes the CSD polypeptide but not the c-Src inhibitor, which is administered separately or vice versa. In other instances, the CSD polypeptide and c-Src inhibitor are each administered to the subject by injection. In a specific embodiment, administration includes intrathecal injection of a pharmaceutical formulation that includes one of the c-Src inhibitor or CSD polypeptide but not the other, which is intrathecally administered in a separate pharmaceutical formulation.

In various embodiments, the opioid is morphine, fentanyl, hydrocodone, hydromorphone, meperidine, oxycontin, oxycodone, or a combination thereof. In a specific embodiment, the opioid is morphine, hydrocodone, oxycontin, oxycodone or a combination thereof. In one embodiment, the opioid therapy is the chronic administration of morphine.

In some embodiments, the CSD polypeptide administered to the subject in accordance with the present methods comprises or consists of the amino acid sequence of SEQ ID NO: 1 or SEQ ID NO: 2, and the c-Src inhibitor administered to the subject is 4-Amino-5-(4-chlorophenyl)-7-(dimethylethyl)pyrazolo[3,4-d]-pyrimidine (PP2) or N-(2-chloro-6-methylphenyl)-2-[[6-[4-(2-hydroxyethyl)piperazin-1-yl]-2-methylpyrimidin-4-yl]amino]-1,3-thiazole-5-carboxamide (dasatinib).

In a specific embodiment, the c-Src inhibitor administered to the subject in accordance with the present methods is PP2 and the CSD polypeptide consists of the amino acid sequence of SEQ ID NO: 1 or SEQ ID NO: 2, or functional fragments of SEQ ID NO: 1 or SEQ ID NO: 2.

In a particular embodiment, the subject is administered chronic opioid therapy, a CSD polypeptide and a c-Src inhibitor intrathecally in accordance with the present methods.

In embodiments, the present disclosure includes a method for reducing opioid analgesic tolerance in a subject treated for chronic pain including: administering to the subject an effective amount of: a caveolin-1 scaffolding domain (CSD) competing polypeptide; and a c-Src inhibitor, wherein the subject has chronic pain and is administered chronic opioid analgesic therapy prior to or concurrently with the administration of the CSD polypeptide and the c-Src inhibitor, and wherein said administration of the CSD polypeptide and the c-Src inhibitor reduces opioid analgesic tolerance in the subject. In some embodiments, the subject is administered the chronic opioid analgesic therapy concurrently with the CSD polypeptide and the c-Src inhibitor, for at least one day prior to the administration of the CSD polypeptide and the c-Src inhibitor, or for at least six days prior to the administration of the CSD polypeptide and the c-Src inhibitor. In some embodiments, the analgesic is an opioid selected from the group consisting of morphine, fentanyl, hydrocodone, hydromorphone, meperidine, oxycontin and oxycodone. In embodiments, the chronic opioid analgesic therapy is administered systemically, intrathecally, or by subcutaneous injection. In some embodiments, the CSD polypeptide and the c-Src inhibitor are administered by spinal injection or intrathecal injection. In some embodiments, the CSD polypeptide comprises or consists of the amino acid sequence of SEQ ID NO:1 or SEQ ID NO:2, or an amino acid sequence having at least 90%, 95%, 97%, or 99% sequence identity to SEQ ID NO:1 or SEQ ID NO:2. In embodiments, the CSD polypeptide may include variants of SEQ ID NO: 1 or SEQ ID NO:2 including 1 or more (several) conservative substitutions. In embodiments, CSD polypeptide includes functional fragments of the amino acid sequence of SEQ ID NO: 1 or SEQ ID NO:2, wherein the functional fragment is capable of competing with Cav1 for binding to Cav1's binding proteins, interrupting Cav1 functionality. In embodiments, CSD polypeptide and/or functional fragments thereof mimic the Cav1 scaffolding domain and competes with Cav1 for binding to signaling molecules thereby interrupting Cav1 scaffolding/signaling.

In embodiments, alterations such as substitutions of amino acids in SEQ ID NOS: 1 or 2 may occur where an L-amino acid is substituted or replaced with a D-amino acid. In another embodiment, peptides may include dextro or a mixture of dextro/levo-amino acids.

In embodiments, the peptides in accordance with the present disclosure may be characterized as cell permeable and include an antennapedia internalization sequence. For example, referring to SEQ ID NO: 1, in embodiments, amino acids in positions 1 to 16 may be characterized as an antennapedia internalization sequence. In embodiments, additional antennapedia internalization sequences may be provided in place of amino acids in positions 1-16 of SEQ ID NO: 1.

In embodiments, the c-Src inhibitor comprises 4-Amino-5-(4-chlorophenyl)-7-(dimethylethyl)pyrazolo[3,4-d]pyrimidine (PP2).

### Embodiments for Preventing the Development of Opioid Analgesic Tolerance

In some embodiments, the present disclosure includes methods for preventing the development of opioid analgesic tolerance. For example, the present disclosure also recognizes that administration of a CSD polypeptide and/or a c-Src inhibitor to a subject with chronic pain can prevent the development of opioid analgesic/antiallodynic tolerance in the subject. In embodiments, a subject who has not yet been administered an opioid analgesic, can be treated with a CSD polypeptide and c-Src inhibitor in order to prohibit the onset of analgesic tolerance.

### Embodiments for Administration of a CSD Polypeptide to Prevent Opioid Analgesic Tolerance

In some embodiments, the present disclosure includes administration of a CSD polypeptide to prevent opioid analgesic tolerance. For example, embodiments of the present disclosure include a method for preventing the development of opioid tolerance in a subject with chronic pain that includes administering an effective amount of a CSD polypeptide to a subject that has not been administered chronic opioid analgesic therapy prior to administration of the CSD polypeptide.

In one embodiment, the method for preventing opioid analgesic tolerance includes administration of a CSD polypeptide to a subject with chronic pain prior to an initial administration of opioid analgesic therapy to the subject. In various embodiments, the subject is administered the CSD polypeptide within 1 day, 2 days, 3 days, 4 days or 5 days prior to initial administration of an opioid analgesic to the subject. In certain embodiments, the subject is administered the CSD polypeptide at least thirty minutes prior to administration of chronic opioid analgesic therapy. In other embodiments, the CSD polypeptide is administered to the subject at least one hour, at least two hours, at least three hours, at least six hours, at least seven hours, at least eight hours, at least nine hours, at least ten hours, at least eleven hours, at least twelve hours, or at least twenty-four hours prior to initial administration of an opioid analgesic therapy. In a certain embodiment, the CSD polypeptide is administered to the subject less than one hour prior to administration of an opioid analgesic therapy.

In other embodiments, the method for preventing opioid analgesic tolerance includes concurrent administration, to the subject, of the CSD polypeptide, and an opioid analgesic.

In various embodiments, the subject is administered the CSD polypeptide prior to the detection of any one of the following: respiratory depression, constipation, immunosuppression, the need for dose escalation of opioid, and hyperalgesia.

As described herein, the method for preventing analgesic tolerance prohibits the development of one or more indicia of opioid-induced analgesic tolerance. Therefore, in some instances, the methods of the present disclosure include administration of a CSD polypeptide prior to detection of one or more indicia of opioid-induced analgesic tolerance in the subject. In a specific embodiment, the subject is administered the CSD polypeptide prior to the detection of the need for dose escalation of opioid analgesic therapy to achieve pain relief in the subject.

In embodiments, the CSD polypeptide and opioid analgesic therapy can be administered in accordance with the methods of the present disclosure for preventing analgesic tolerance in any amount and route of administration described above, the entire contents of which are incorporated herein by reference.

As described above, the opioid analgesic therapy can be administered to the subject systemically or intrathecally. In some embodiments, the opioid analgesic therapy is administered to the subject systemically, such as by oral administration, topical administration, subcutaneous implantation or by intravenous injection. In a specific embodiment, the opioid is morphine, and the opioid analgesic therapy is administered to the subject by subcutaneous release of morphine pellets.

In various embodiments, the opioid analgesic therapy is administered to the subject by spinal injection or intrathecally, such as by injection to the spinal cord of the subject. In a specific embodiment, the opioid is morphine, and the opioid analgesic therapy is administered to the subject by intrathecal injection.

In some embodiments, the method includes administration of the CSD polypeptide systemically by spinal injection, or intrathecally. In certain instances, the CSD polypeptide is administered by injection.

In a specific embodiment, the CSD polypeptide is administered to the subject intrathecally.

As stated above, the CSD polypeptide and opioid analgesic therapy can be administered together or separately through the same mode of administration or separate modes of administration. In various embodiments, the CSD polypeptide and opioid are formulated for co-administration in a single or separate injection, topical formulation or hard or soft gelatin capsule, pill, tablet, including a coated tablet, elixir, suspensions, syrup or inhalable. In certain embodiments, the CSD polypeptide and opioid are formulated for co-administration in a single dose. In one embodiment, the opioid and CSD polypeptide are administered to the subject intrathecally, as a single injection to the spinal cord of the subject.

In other instances, the CSD polypeptide can be administered by injection and the opioid can be administered to the subject subcutaneously, or vice versa. In some instances, administration includes administering, to the subject, a pharmaceutical formulation that includes the CSD polypeptide but not an opioid, which is administered separately or vice versa. In other instances, the CSD polypeptide and opioid are each administered to the subject by injection. In a specific embodiment, administration includes intrathecal injection of a pharmaceutical formulation that includes one of an opioid or CSD polypeptide but not the other, which is intrathecally administered in a separate pharmaceutical formulation.

In various embodiments, the opioid analgesic therapy includes administration of morphine, fentanyl, hydrocodone, hydromorphone, meperidine, oxycontin, oxycodone, or a combination thereof. In a specific embodiment, the opioid analgesic therapy includes morphine, hydrocodone, oxycontin, oxycodone, or a combination thereof. In one embodiment, the opioid analgesic therapy includes administration of chronic morphine to the subject.

In some embodiments, the CSD polypeptide administered to the subject in accordance with the present methods comprises the amino acid sequence of SEQ ID NO: 1, variant, or functional fragment thereof. In some embodiments, the CSD polypeptide administered to the subject in accordance with the present methods comprises the amino acid sequence of SEQ ID NO: 2, variant, or functional fragment thereof.

In a particular embodiment, the subject is intrathecally administered chronic opioid therapy, and a CSD polypeptide in accordance with the present methods in order to prevent the development of opioid-induced tolerance in the subject.

In a specific embodiment, the opioid analgesic therapy administered to the subject in accordance with the present methods is morphine, and the CSD polypeptide administered consists of the amino acid sequence of SEQ ID NO: 1 or SEQ ID NO:2.

In some embodiments, the present disclosure includes a method for preventing opioid analgesic tolerance in a subject with chronic pain including: administering, to the subject, an effective amount of a caveolin-1 scaffolding domain (CSD) competing polypeptide, wherein the subject has chronic pain and has not been administered chronic opioid analgesic therapy prior to said administration of the CSD polypeptide. In some embodiments, the methods further include administering chronic opioid analgesic therapy to the subject concurrently with the CSD polypeptide, after the administration of the CSD polypeptide, at least 30 minutes after the administration of the CSD polypeptide, or at least 1 day after the administration of the CSD polypeptide. In some embodiments, the chronic opioid analgesic therapy is administered systemically or intrathecally. In embodiments, the analgesic is an opioid selected from the group consisting of morphine, sufentanil, hydrocodone, hydromorphone, meperidine, oxycontin, oxycodone, and combinations thereof. In embodiments, the CSD polypeptide is administered orally, by injection, or by intrathecal injection. In embodiments, the CSD polypeptide comprises or consists of the amino acid sequence of SEQ ID NO:1 or SEQ ID NO: 2, or an amino acid sequence having at least 90%, 95%, 97%, or 99% sequence identity to SEQ ID NO: 1 or SEQ ID NO:2. In embodiments, the CSD polypeptide may include variants of SEQ ID NO: 1 or SEQ ID NO:2 including 1 or more (several) conservative substitutions. In embodiments, CSD polypeptide includes functional fragments of the amino acid sequence of SEQ ID NO: 1 or SEQ ID NO:2, wherein the functional fragment is capable of competing with Cav1 for binding to Cav1's binding proteins, interrupting Cav1 functionality. In embodiments, CSD polypeptide and/or functional fragments thereof mimic the Cav1 scaffolding domain and competes with Cav1 for binding to signaling molecules thereby interrupting Cav1 scaffolding/signaling.

### Embodiments for Administration of a C-Src Inhibitor to Prevent Opioid Analgesic Tolerance

In embodiments, the present disclosure includes the administration of a c-Src inhibitor to prevent opioid analgesic tolerance. For example, the present disclosure includes administering a c-Src inhibitor alone to a subject with chronic pain in order to prevent opioid analgesic tolerance development or onset in the subject. In this instance, the inventive method for preventing the development of opioid tolerance in a subject with chronic pain that includes administering an effective amount of a c-Src inhibitor to a subject that has not been administered chronic opioid analgesic therapy prior to administration of the c-Src inhibitor. In some embodiments, the method of the present disclosure for preventing the development of opioid tolerance in a subject with chronic pain that includes administering an effective amount of a c-Src inhibitor to a subject that has not been administered opioid analgesic therapy prior to administration of the c-Src inhibitor. In embodiments, opioid analgesic therapy is initiated after administering an effective amount of a c-Src inhibitor to a subject.

In one embodiment, the method for preventing opioid analgesic tolerance includes administration of a c-Src inhibitor to a subject with chronic pain prior to an initial administration of opioid analgesic therapy to the subject. In various embodiments, the subject is administered the c-Src inhibitor within 1 day, 2 days, 3 days, 4 days or 5 days of initial administration of an opioid analgesic to the subject. In certain embodiments, the subject is administered the c-Src inhibitor at least thirty minutes prior to administration of chronic opioid analgesic therapy. In other embodiments, the c-Src inhibitor is administered to the subject at least one hour, at least two hours, at least three hours, at least four hours, at least five hours, at least six hours, at least seven hours, at least eight hours, at least nine hours, at least ten hours, at least eleven hours, at least twelve hours, or at least twenty-four hours prior to initial administration of an opioid analgesic therapy. In embodiments, the c-Src inhibitor is administered to the subject less than one hour prior to administration of an opioid analgesic therapy.

In various embodiments, the subject is administered the c-Src inhibitor prior to the detection of any one of the following disorders: respiratory depression, constipation, immunosuppression, the need for opioid dose escalation, and hyperalgesia.

As described herein, the method for preventing analgesic tolerance prohibits the development of one or more indicia of opioid-induced analgesic tolerance. Therefore, in some instances, the methods of the present disclosure include administration of a c-Src inhibitor prior to detection of one or more indicia of opioid-induced analgesic tolerance in the subject. In a specific embodiment, the subject is administered the c-Src inhibitor prior to the detection of the need for dose escalation of opioid analgesic therapy to achieve pain relief in the subject.

In other embodiments, the method for preventing opioid analgesic tolerance includes concurrent administration, to the subject, of the c-Src inhibitor, and an opioid analgesic therapy.

The c-Src inhibitor and opioid analgesic therapy can be administered in accordance with the inventive method for preventing analgesic tolerance in any amount and route of administration described above, the entire contents of which are incorporated herein by reference.

As described above, the opioid analgesic therapy can be administered to the subject systemically or intrathecally. In some embodiments, the opioid analgesic therapy is administered to the subject systemically, such as by oral administration, topical administration, subcutaneous implantation or by intravenous injection. In a specific embodiment, the opioid is morphine, and the opioid analgesic therapy is administered to the subject by subcutaneous release of morphine pellets.

In various embodiments, the opioid analgesic therapy is administered to the subject intrathecally, such as by injection to the spinal cord of the subject. In a specific embodiment, the opioid is morphine, and the opioid analgesic therapy is administered to the subject by intrathecal injection.

In some embodiments, the method includes administration of the c-Src inhibitor systemically or intrathecally. In certain instances, the c-Src inhibitor is administered by injection.

In a specific embodiment, the c-Src inhibitor is administered to the subject intrathecally.

As stated above, the c-Src inhibitor and opioid analgesic therapy can be administered together or separately through the same mode of administration or separate modes of administration. In various embodiments, the opioid analgesic therapy and c-Src inhibitor are formulated for co-administration in a single or separate injection, topical formulation or hard or soft gelatin capsule, pill, tablet, including a coated tablet, elixir, suspensions, syrup or inhalable. In certain embodiments, the opioid and c-Src inhibitor are formulated for co-administration in a single dose. In one embodiment, the c-Src inhibitor and opioid analgesic are administered to the subject intrathecally, as a single injection to the spinal cord of the subject.

In other instances, the c-Src inhibitor can be administered by injection and the opioid analgesic can be administered to the subject subcutaneously, or vice versa. In some instances, administration includes administering, to the subject, a pharmaceutical formulation that includes an opioid but not a c-Src inhibitor, which is administered separately, or vice versa. In other instances, the opioid and c-Src inhibitor are each administered to the subject by injection. In a specific embodiment, administration includes intrathecal injection of a pharmaceutical formulation that includes one of the c-Src inhibitor or an opioid but not the other, which is intrathecally administered in a separate pharmaceutical formulation.

In some embodiments, the c-Src inhibitor administered to the subject is 4-Amino-5-(4-chlorophenyl)-7-(dimethylethyl)pyrazolo[3,4-d]-pyrimidine (PP2), N-(2-chloro-6-methylphenyl)-2-[[6-[4-(2-hydroxyethyl)piperazin-1-yl]-2-methylpyrimidin-4-yl]amino]-1,3-thiazole-5-carboxamide (dasatinib), or a combination thereof. In a specific embodiment, the c-Src inhibitor administered to the subject is PP2.

In a particular embodiment, the subject is intrathecally administered chronic opioid therapy, and a c-Src inhibitor in accordance with the present methods in order to prevent the development of opioid-induced tolerance in the subject. In a specific embodiment, the opioid analgesic therapy administered to the subject in accordance with the present methods is morphine, and the c-Src inhibitor administered is PP2.

In some embodiments, the present disclosure includes a method for preventing opioid analgesic tolerance in a subject with chronic pain including: administering, to the subject, an effective amount of a c-Src inhibitor, wherein the subject has chronic pain and has not been administered chronic opioid analgesic therapy prior to the administration of the c-Src inhibitor. In embodiments, the method further includes administering chronic opioid analgesic therapy to the subject concurrently with the c-Src inhibitor, after the administration of the c-Src inhibitor, at least 30 minutes after the administration of the c-Src inhibitor, or at least 1 day after the administration of the c-Src inhibitor. In embodiments, the chronic opioid analgesic therapy is administered systemically or intrathecally. In embodiments, the analgesic is an opioid selected from the group consisting of morphine, sufentanil, hydrocodone, hydromorphone, meperidine, oxycontin, oxycodone, and combinations thereof. In embodiments, the c-Src inhibitor is administered by injection, or by intrathecal injection. In embodiments, the c-Src inhibitor comprises 4-Amino-5-(4-chlorophenyl)-7-(dimethylethyl)pyrazolo[3,4-d]pyrimidine (PP2). In embodiments, the subject has neuropathic chronic pain.

### Embodiments for Co-Administration of a CSD Polypeptide and a C-Src Inhibitor to Prevent Opioid Analgesic Tolerance

In another aspect of the present disclosure, a method for preventing the development of opioid tolerance in a subject with chronic pain is presented that includes administering an effective amount of a CSD polypeptide and a c-Src inhibitor to a subject that has not been administered chronic opioid analgesic therapy prior to administration of the CSD polypeptide and c-Src inhibitor. In embodiments, opioid analgesic therapy is initiated after administration of CSD polypeptide and c-Src inhibitor.

In one embodiment, the method for preventing opioid analgesic tolerance includes administration of a CSD polypeptide and a c-Src inhibitor to a subject with chronic pain prior to an initial administration of opioid analgesic therapy to the subject. In various embodiments, the subject is administered the CSD polypeptide and a c-Src inhibitor within 1 day, 2 days, 3 days, 4 days or 5 days prior to the initial administration of an opioid analgesic to the subject. In certain embodiments, the subject is administered the CSD polypeptide and c-Src inhibitor at least thirty minutes prior to administration of chronic opioid analgesic therapy. In other embodiments, the CSD polypeptide and c-Src inhibitor are administered to the subject at least one hour, at least two hours, at least three hours, at least four hours, at least five hours, at least six hours, at least seven hours, at least eight hours, at least nine hours, at least ten hours, at least eleven hours, at least twelve hours, or at least twenty-four hours prior to initial administration of an opioid analgesic therapy. In certain embodiments, the CSD polypeptide and c-Src inhibitor are administered to the subject less than one hour prior to administration of an opioid analgesic therapy.

In various embodiments, the subject is administered the CSD polypeptide and c-Src inhibitor prior to the detection of any one of the following disorders: respiratory depression, constipation, immunosuppression, the need for opioid dose escalation, and hyperalgesia.

As described herein, the method for preventing analgesic tolerance prohibits the development of one or more indicia of opioid-induced analgesic tolerance. Therefore, in some instances, the methods of the present disclosure include administration of a CSD polypeptide and c-Src inhibitor prior to detection of one or more indicia of opioid-induced analgesic tolerance in the subject. In a specific embodiment, the subject is administered the CSD polypeptide and c-Src inhibitor prior to the detection of the need for dose escalation of opioid analgesic therapy to achieve pain relief in the subject.

In other embodiments, the method for preventing opioid analgesic tolerance includes concurrent administration, to the subject, of the CSD polypeptide, the c-Src inhibitor and an opioid analgesic. The CSD polypeptide, c-Src inhibitor and opioid analgesic therapy can be administered in accordance with the inventive method for preventing analgesic tolerance in any amount and route of administration described above, the entire contents of which are incorporated herein by reference.

As described above, the opioid analgesic therapy can be administered to the subject systemically, by spinal injection, or intrathecally. In some embodiments, the opioid analgesic therapy is administered to the subject systemically, such as by oral administration, topical administration, subcutaneous implantation or by intravenous injection. In a specific embodiment, the opioid is morphine, and the opioid analgesic therapy is administered to the subject by subcutaneous release of morphine pellets.

In various embodiments, the opioid analgesic therapy is administered to the subject intrathecally, such as by injection to the spinal cord of the subject. In a specific embodiment, the opioid is morphine, and the opioid analgesic therapy is administered to the subject by intrathecal injection.

In some embodiments, the method includes administration of the CSD polypeptide and c-Src inhibitor systemically, by spinal injection or intrathecally. In certain instances, the CSD polypeptide and c-Src inhibitor are administered by injection.

In a specific embodiment, the CSD polypeptide and c-Src inhibitor are administered to the subject intrathecally.

As stated above, the CSD polypeptide, c-Src inhibitor and opioid analgesic therapy can be administered together or separately through the same mode of administration or separate modes of administration. In various embodiments, the CSD polypeptide and/or the c-Src inhibitor, and opioid are formulated for co-administration in a single or separate injection, topical formulation or hard or soft gelatin capsule, pill, tablet, including a coated tablet, elixir, suspensions, syrup or inhalable. In certain embodiments, the CSD polypeptide and/or c-Src inhibitor, and opioid are formulated for co-administration in a single dose. In one embodiment, the opioid, CSD polypeptide and c-Src inhibitor are administered to the subject intrathecally, as a single injection to the spinal cord of the subject.

In other instances, the CSD polypeptide and c-Src inhibitor can be administered by injection and the opioid can be administered to the subject subcutaneously, or vice versa. In some instances, administration includes administering, to the subject, a pharmaceutical formulation that includes the CSD polypeptide and/or the c-Src inhibitor but not an opioid, which is/are administered separately or vice versa. In other instances, the CSD polypeptide, c-Src inhibitor and opioid are each administered to the subject by injection. In a specific embodiment, administration includes intrathecal injection of a pharmaceutical formulation that includes one of an opioid or CSD polypeptide or c-Src inhibitor but not a combination thereof, each remaining composition is then intrathecally administered in a separate pharmaceutical formulation.

In some embodiments, the CSD polypeptide administered to the subject in accordance with the present methods comprises or consists of the amino acid sequence of SEQ ID NO: 1 or SEQ ID NO: 2, and the c-Src inhibitor administered to the subject is 4-Amino-5-(4-chlorophenyl)-7-(dimethylethyl)pyrazolo[3,4-d]-pyrimidine (PP2) or N-(2-chloro-6-methylphenyl)-2-[[6-[4-(2-hydroxyethyl)piperazin-1-yl]-2-methylpyrimidin-4-yl]amino]-1,3-thiazole-5-carboxamide (dasatinib).

In various embodiments, the opioid analgesic therapy includes administration of morphine, fentanyl, hydrocodone, hydromorphone, meperidine, oxycontin, oxycodone, or a combination thereof. In a specific embodiment, the opioid analgesic therapy includes morphine, hydrocodone, oxycontin, oxycodone, or a combination thereof. In one embodiment, the opioid analgesic therapy includes administration of chronic morphine to the subject.

In a specific embodiment, the opioid analgesic therapy administered to the subject in accordance with the present methods is morphine, the c-Src inhibitor administered is PP2 and the CSD polypeptide administered consists of the amino acid sequence of SEQ ID NO: 1, or SEQ ID NO: 2.

In a particular embodiment, the subject is intrathecally administered chronic opioid therapy, a CSD polypeptide and a c-Src inhibitor in accordance with the present methods in order to prevent the development of opioid-induced tolerance in the subject.

In embodiments the present disclosure includes a method for preventing opioid analgesic tolerance in a subject with chronic pain comprising: administering to the subject an effective amount of: a caveolin-1 scaffolding domain (CSD) polypeptide; and a c-Src inhibitor, wherein the subject has chronic pain and has not been administered chronic opioid analgesic therapy prior to said administration of the CSD polypeptide and the c-Src inhibitor. In embodiments, the methods may further include administering chronic opioid analgesic therapy to the subject concurrently with the CSD polypeptide and the c-Src inhibitor, after the administration of the CSD polypeptide and the c-Src inhibitor, at least 30 minutes after the administration of the CSD polypeptide and the c-Src inhibitor, or at least 1 day after the administration of the CSD polypeptide and the c-Src inhibitor. In some embodiments, the chronic opioid analgesic therapy is administered systemically or intrathecally. In some embodiments, the analgesic is an opioid selected from the group consisting of morphine, sufentanil, hydrocodone, hydromorphone, meperidine, oxycontin and oxycodone. In some embodiments, the CSD polypeptide and the c-Src inhibitor are administered by injection, or by intrathecal application. In some embodiments, the CSD polypeptide comprises the amino acid sequence set forth in SEQ ID NO:1 or SEQ ID NO: 2, or an amino acid sequence having at least 90%, 95%, 97%, or 99% sequence identity to SEQ ID NO: 1 or to SEQ ID NO: 2. In embodiments, the CSD polypeptide may include variants of SEQ ID NO: 1 or SEQ ID NO: 2, including 1, 2, 3, 4, or more (several) conservative substitutions. In embodiments, CSD polypeptide includes functional fragments of the amino acid sequence of SEQ ID NO: 1, wherein the functional fragment is capable of competing with Cav1 for binding to Cav1's binding proteins, interrupting Cav1 functionality. In embodiments, the c-Src inhibitor comprises or consists of 4-Amino-5-(4-chlorophenyl)-7-(dimethylethyl)pyrazolo[3,4-d]pyrimidine (PP2).

### Embodiments for Treating, Ameliorating, or Preventing Pain

In some embodiments, the present disclosure relates to a method of treating, ameliorating, or preventing one or more symptoms of pain in a subject, including: administering a therapeutically effective amount of an opioid to a subject; and administering a therapeutically effective amount of a polypeptide having at least 90%, 95%, 97%, or 99% sequence identity to SEQ ID NO: 1 or to SEQ ID NO: 2, a c-Src inhibitor, or a polypeptide having at least 90%, 95%, 97%, or 99% sequence identity to SEQ ID NO: 1 or SEQ ID NO: 2, and a c-Src inhibitor to a subject in need thereof. In some embodiments, administering a therapeutically effective amount of an opioid to a subject is administered prior to or concurrently with the administration of a polypeptide having at least 90% sequence identity to SEQ ID NO: 1 or a polypeptide having at least 90%, 95%, 97%, or 99% sequence identity to SEQ ID NO: 2. In some embodiments, the therapeutically effective amount of a polypeptide having at least 90%, 95%, 97%, or 99% sequence identity to SEQ ID NO: 1 or at least 90%, 95%, 97%, or 99% sequence identity to SEQ ID NO: 2 is an amount sufficient to reduce an analgesic tolerance in the subject. In some embodiments, the variants of the polypeptide of SEQ ID NO: 1 or SEQ ID NO: 2 may include highly similar sequences including sequences having one or more, or several conservatively substituted amino acids, such as where amino acid is exchanged with another amino acid having similar properties. Non-limiting examples of conservative substitutions include amino acid substitutions that do not generally alter specific activity which are described, for example, by H. Neurath and R. L. Hill, 1979, In, The Proteins, Academic Press, New York. Common substitutions are Ala/Ser, Val/Ile, Asp/Glu.

In some embodiments, the present disclosure includes a method of treating, ameliorating, or preventing one or more symptoms of pain in a subject, including: administering a therapeutically effective amount of an opioid to a subject; and administering a therapeutically effective amount of a polypeptide having at least 90% sequence identity to SEQ ID NO: 2, a non-receptor tyrosine kinase (c-Src) inhibitor, or a polypeptide having at least 90% sequence identity to SEQ ID NO: 2 and a c-Src inhibitor to a subject in need thereof. In some embodiments, administering a therapeutically effective amount of an opioid to a subject is performed prior to or concurrently with the administration of a polypeptide having at least 90% sequence identity to SEQ ID NO: 2. In some embodiments, the therapeutically effective amount of a polypeptide having at least 90% sequence identity to SEQ ID NO: 2 is an amount sufficient to reduce an analgesic tolerance in the subject. In some embodiments, the polypeptide having at least 90% sequence identity to SEQ ID NO: 2 has at least 95%, at least 97%, or at least 99% sequence identity to SEQ ID NO:2. In some embodiments, the present disclosure relates to a method for increasing the activity of an analgesic in a subject including: administering, to the subject, an effective amount of: a caveolin-1 scaffolding domain (CSD) polypeptide; and a c-Src inhibitor, wherein the subject has been administered an analgesic for at least 1 day prior to the administration of the CSD and c-Src inhibitor, and wherein said administration of the CSD and c-Src inhibitor increases the analgesic activity in the subject. In embodiments, the subject has been administered the analgesic for at least three days prior to the administration of the CSD and c-Src inhibitor. In embodiments, the subject has been administered the analgesic for at least six days prior to the administration of the CSD and c-Src inhibitor. In embodiments, the subject has been administered the analgesic for at least ten days prior to the administration of the CSD and c-Src inhibitor. In embodiments, the analgesic is an opioid. In embodiments, the opioid is selected from the group consisting of morphine, sufentanil, hydrocodone and oxycodone. In embodiments, the analgesic is administered systemically. In embodiments, the CSD and c-Src inhibitor is administered systemically. In embodiments, the CSD and c-Src inhibitor is administered orally or by injection. In embodiments, the CSD polypeptide comprises the amino acid sequence (DGIWKASFTTFTVTKYWFYR) (SEQ ID NO:2). In embodiments, the c-Src inhibitor consists of or comprises 4-Amino-5-(4-chlorophenyl)-7-(dimethylethyl)pyrazolo[3,4-d]pyrimidine (PP2).

In some embodiments, the present disclosure includes a method for preventing analgesic tolerance in a subject including: administering, to the subject, an effective amount of: a caveolin-1 scaffolding domain (CSD) polypeptide; and a c-Src inhibitor, wherein the subject has not been administered an analgesic prior to said administration of the CSD and c-Src inhibitor. In embodiments, the analgesic is an opioid. In embodiments, the opioid is selected from the group consisting of morphine, sufentanil, hydrocodone and oxycodone. In embodiments, the CSD and c-Src inhibitor are administered systemically. In embodiments, the CSD and c-Src inhibitor are administered orally or by injection. In embodiments, the CSD polypeptide comprises the amino acid sequence (DGIWKASFTTFTVTKYWFYR) (SEQ ID NO:2).

In embodiments, the c-Src inhibitor comprises or consists of 4-Amino-5-(4-chlorophenyl)-7-(dimethylethyl)pyrazolo[3,4-d]pyrimidine (PP2). In embodiments, the method further includes administering analgesic to the subject after the administration of the CSD and c-Src inhibitor. In embodiments, the analgesic is administered at least 30 minutes prior to the administration of the CSD and c-Src inhibitor. In embodiments, the analgesic is administered at least 1 day prior to the administration of the CSD and c-Src inhibitor.

In some embodiments, the present disclosure includes a method for preventing and/or reducing opioid analgesic tolerance in a subject treated for chronic pain including: administering, to the subject, an effective amount of a caveolin-1 scaffolding domain (CSD) polypeptide, wherein the subject has chronic pain and is administered chronic opioid analgesic therapy prior to or concurrently with the administration of the CSD polypeptide, and wherein said administration of the CSD polypeptide reduces analgesic tolerance in the subject. In embodiments, the subject is administered the chronic opioid analgesic therapy concurrently with the CSD polypeptide, for at least one day prior to the administration of the CSD polypeptide, for at least six days prior to the administration of the CSD polypeptide, or the subject has not been administered opioid analgesic therapy prior to the administration of the CSD polypeptide.

In some embodiments, the present disclosure includes a method for preventing and/or reducing opioid analgesic tolerance in a subject treated for chronic pain including: administering to the subject an effective amount of a non-receptor tyrosine kinase (c-Src) inhibitor, wherein the subject has chronic pain and is administered chronic opioid analgesic therapy prior to or concurrently with the administration of the c-Src inhibitor, and wherein said administration of the c-Src inhibitor reduces opioid analgesic tolerance in the subject. In embodiments, the subject is administered the chronic opioid analgesic therapy concurrently with the c-Src inhibitor, for at least one day prior to the administration of the c-Src inhibitor, for at least six days prior to the administration of the c-Src inhibitor, or the subject has not been administered opioid analgesic therapy prior to the administration of the c-Src inhibitor.

In some embodiments, the present disclosure relates to a method for preventing and/or reducing opioid analgesic tolerance in a subject treated for chronic pain including: administering to the subject an effective amount of: a caveolin-1 scaffolding domain (CSD) polypeptide and a non-receptor tyrosine kinase (c-Src) inhibitor, wherein the subject has chronic pain and is administered chronic opioid analgesic therapy prior to or concurrently with the administration of the CSD polypeptide and the c-Src inhibitor, and wherein said administration of the CSD polypeptide and the c-Src inhibitor reduces opioid analgesic tolerance in the subject. In embodiments, the subject is administered the chronic opioid analgesic therapy concurrently with the CSD polypeptide and the c-Src inhibitor, for at least one day prior to the administration of the CSD polypeptide and the c-Src inhibitor, for at least six days prior to the administration of the CSD polypeptide and the c-Src inhibitor, or the subject has not been administered opioid analgesic therapy prior to the administration of the CSD polypeptide and the c-Src inhibitor.

### Use in Treating Subjects

Embodiments of the present disclosure are suitable for use as a medicament for use in pain management and/or for use in the treatment of subjects for pain such as chronic pain. In embodiments, the present disclosure includes substances (caveolin-1 scaffolding domain (CSD) polypeptide, c-Src inhibitor, and/or opioid), or a pharmaceutically acceptable salt or ester thereof, for use as a medicament for use in the treatment of pain.

In embodiments, the present disclosure includes the substance caveolin-1 scaffolding domain (CSD) polypeptide, or a pharmaceutically acceptable salt or ester thereof, for use as a medicament for use in the treatment of pain.

In embodiments, the present disclosure includes the substance c-Src inhibitor, or a pharmaceutically acceptable salt or ester thereof, for use as a medicament for use in the treatment of pain.

In embodiments, the present disclosure includes the substances caveolin-1 scaffolding domain (CSD) polypeptide, or a pharmaceutically acceptable salt or ester thereof, and opioid, or a pharmaceutically acceptable salt or ester thereof, for use as a medicament for use in the treatment of pain.

In embodiments, the present disclosure includes the substances c-Src inhibitor or a pharmaceutically acceptable salt or ester thereof, and opioid or a pharmaceutically acceptable salt or ester thereof, for use as a medicament for use in the treatment of pain.

In embodiments, the present disclosure includes substances (caveolin-1 scaffolding domain (CSD) polypeptide, c-Src inhibitor, and/or opioid or pharmaceutically acceptable salts or esters thereof) for use as a medicament for use in the treatment of opioid tolerance in humans.

In embodiments, the present disclosure includes the substance caveolin-1 scaffolding domain (CSD) polypeptide or a pharmaceutically acceptable salt or ester thereof, for use in the treatment of opioid tolerance in humans.

In embodiments, the present disclosure includes the substance c-Src inhibitor or a pharmaceutically acceptable salt or ester thereof, for use as a medicament for use in the treatment of opioid tolerance in humans.

In embodiments, the present disclosure includes the substances caveolin-1 scaffolding domain (CSD) polypeptide or a pharmaceutically acceptable salt or ester thereof, and opioid or a pharmaceutically acceptable salt or ester thereof, for use in the treatment of opioid tolerance in humans.

In embodiments, the present disclosure includes the substances c-Src inhibitor or a pharmaceutically acceptable salt or ester thereof, and opioid or a pharmaceutically acceptable salt or ester thereof, for use as a medicament for use in the treatment of opioid tolerance in humans.

In embodiments, the present disclosure includes substances (caveolin-1 scaffolding domain (CSD) polypeptide, c-Src inhibitor, and/or opioid or pharmaceutically acceptable salts or esters thereof) for use as a medicament for use in reducing or preventing opioid tolerance in humans.

In embodiments, the present disclosure includes the substance caveolin-1 scaffolding domain (CSD) polypeptide or a pharmaceutically acceptable salt or ester thereof, for use in reducing or preventing opioid tolerance in humans.

In embodiments, the present disclosure includes the substance c-Src inhibitor or a pharmaceutically acceptable salt or ester thereof, for use as a medicament for use in reducing or preventing opioid tolerance in humans.

In embodiments, the present disclosure includes the substances caveolin-1 scaffolding domain (CSD) polypeptide or a pharmaceutically acceptable salt or ester thereof, and opioid or a pharmaceutically acceptable salt or ester thereof, for use in reducing or preventing opioid tolerance in humans.

In embodiments, the present disclosure includes the substances c-Src inhibitor or a pharmaceutically acceptable salt or ester thereof, and opioid or a pharmaceutically acceptable salt or ester thereof, for use as a medicament for use in reducing or preventing opioid tolerance in humans.

In some embodiments, a caveolin-1 scaffolding domain (CSD) polypeptide or a pharmaceutically acceptable salt or ester thereof, c-Src inhibitor or a pharmaceutically acceptable salt or ester thereof, and/or opioid or a pharmaceutically acceptable salt or ester thereof, is provided for use as a medicament for use in the treatment of chronic pain. In embodiments, the chronic pain is from one or more disorders such as fibromyalgia, rheumatoid arthritis, osteoarthritis, chronic arthropathy, spinal nerve compression syndromes associated with neoplasia and/or disc heriation, chronic joint pain of any etiology associated with inflammation and/or structural joint abnormalities, post herpetic neuralgia, trigeminal neuralgia, chronic metabolic neuropathy associated with chronic pain, migraine, inflammatory pain, post-surgical pain syndromes, irritable bowel syndrome, autonomic neuropathies, and chronic pain syndrome associated with activation of central sensitization pathways.

In embodiments, the present disclosure includes substances (caveolin-1 scaffolding domain (CSD) polypeptide, and/or c-Src inhibitor, or pharmaceutically acceptable salts or esters thereof) for use as a medicament to prevent antiallodynic and/or antinociceptive tolerance development to chronic systemic opioid administration.

### EXAMPLES

The following examples further illustrate the methods of the disclosure but should not be construed to limit the scope of the disclosure in any way.

### Example 1. Methods and Materials

Rats. Experiments utilized rats (Charles River Laboratories, Kingston, N.Y.; 230-300 g) that were maintained in a controlled environment on a 12-hour light/dark cycle. Food and water were available ad libitum. Animals were randomly assigned to experimental and control groups. Power analysis guided experimental group size, which is indicated in relevant sections of Results.

Spinal nerve ligation (SpNL). Under isoflurane anesthesia (2.5%), an incision was made above the lumbar spine and the left transverse process of vertebra L6 was removed in order to expose the underneath L5 spinal nerve. The left L5 spinal nerve was tightly ligated with silk thread No. 6 and cut distal to the ligation. Peripheral neuropathic pain emerges in the ipsilateral hind paws, manifested as mechanical allodynia within 24 h, which remains stable for months (mechanical allodynic thresholds are unaffected on the contralateral side). General behavior of the rats was monitored before and after the surgery. Rats showing difficulty elevating the hind paw ipsilateral to SpNL were excluded.

Implantation of Intrathecal Cannulas. A permanent indwelling cannula was inserted into the lumbar spinal cord subarachnoid space as we have described and utilized previously for spinal drug delivery. See e.g., Storman, E. M., et al., Physical Linkage of Estrogen Receptor alpha and Aromatase in Rat: Oligocrine and Endocrine Actions of CNS-Produced Estrogens. Endocrinology, 2018. 159(7): p. 2683-2697; Liu, N.J., et al., Estrogens synthesized and acting within a spinal oligomer suppress spinal endomorphin 2 antinociception: ebb and flow over the rat reproductive cycle. Pain, 2017. 158(10): p. 1903-1914; and Gintzler, A. R. and N. J. Liu, Harnessing endogenous opioids for pain relief: Fantasy vs reality. J Opioid Manag, 2019. 16(1): p. 67-72.) Spinal cannula was implanted concomitant with SpNL. Briefly, animals were anesthetized as described above, and an 8.0 cm PE-10 catheter (Becton Dickinson and Company, Franklin Lanes, N.J.) was inserted into the spinal subarachnoid space via the atlanto-occipital membrane. The cephalic portion of the catheter was externalized through the skin on the dorsal side of the neck, where it was secured in place, being relatively inaccessible to the paws. All animals utilized in studies appeared to be free of infection upon gross inspection and did not exhibit any motoric impairment, assessed using the righting reflex and the inclined plane test.

Intrathecal Administration of Drugs. Drugs were applied to the spinal cord subarachnoid space over a 60-second period via the indwelling intrathecal (i.t.) cannula seven days following cannula implantation. Flushing the cannula with an additional 10 μl of saline ensured complete delivery. I.t. pretreatment with either caveolin-1 scaffolding domain competing peptide (CSD), scrambled caveolin-1 scaffolding domain competing peptide (S-CSD), PP2, PP3 or CSD+PP2 was applied 45 min before i.t. application of morphine. I.t. morphine dose responsiveness was determined 30 min after each i.t. application of morphine, immediately before the application of the following dose of morphine. (See also e.g., Pelissier, T., et al., Analgesia is produced by intrathecal administration of the kappa opioid agonist, U50,488H, on formalin-evoked cutaneous pain in the rat. Eur. J. Pharmacol., 1990. 190: p. 287-293; Sullivan, A. F. and A. H. Dickenson, Electrophysiologic studies on the spinal antinociceptive action of kappa opioid agonist in the adult and 21-day old rat. J. Pharmacol. Exp. Ther., 1991. 256: p. 1119-1125; Dawson-Basoa, M. E. and A. R. Gintzler, Estrogen and Progesterone activate spinal kappa-opiate receptor analgesic mechanisms. Pain, 1996. 64: p. 607-615.) Dose responsiveness was always performed in the identical ascending pattern among the various experimental groups, minimizing any potential confounds resulting from differences in duration of action among the various ascending doses of i.t. morphine.

Chronic Morphine Administration Generating Spinal or Systemic Morphine Tolerance. Spinal morphine tolerance was achieved via i.t. application of 15 μg morphine daily for at least six days (See e.g., Powell, K. J., et al., Blockade and reversal of spinal morphine tolerance by peptide and non-peptide calcitonin gene-related peptide receptor antagonists. Br J Pharmacol, 2000. 131(5): p. 875-84 and Liu, N.-J., S. Chakrabarti, and A. R. Gintzler, Chronic morphine-induced loss of the facilitative interaction between vasoactive intestinal polypeptide and delta-opioid: involvement of protein kinase C and and phospholipase Cbs. Brain Res, 2004. 1010: p. 1-9.) Systemic morphine tolerance was accomplished by implanting morphine pellet subcutaneously under isoflurane anesthesia (2.5%), one pellet on day 1, two pellets on day 3 and three pellets on day 5 (each containing 75 mg morphine base) (See e.g., Chakrabarti, S., et al., Chronic opioid treatment augments caveolin-1 scaffolding: relevance to stimulatory mu-opioid receptor adenylyl cyclase signaling. J Neurochem, 2016. 139(5): p. 737-747 and Bhargava, H. N. and V. M. Villar, Tolerance-dependence and serum elimination of morphine in rats implanted with morphine pellets. Gen Pharmacol, 1991. 22(6): p. 1033-42.)

Quantification of Allodynia and Acute Thermal Nocicepton. Mechanical allodynia was quantified by measuring withdrawal thresholds of the hind paw ipsilateral to the SpNL in response to the application of von Frey force as described previously (See e.g., Liu, N. J., E. M. Storman, and A. R. Gintzler, Pharmacological Modulation of Endogenous Opioid Activity to Attenuate Neuropathic Pain in Rats. J Pain, 2019. 20(2): p. 235-243.) Briefly, rats were placed on a wire mesh surface, covered by an inverted plastic cage and allowed to habituate for 15 min. A hand-held probe containing a rigid filament was applied to the plantar surface of the hind paw with increasing force. The applied pressure (g) that elicited paw withdrawal was automatically recorded using Electronic von Frey Anesthesiometer (IITC Life Science, Woodland Hills, Calif.). A cutoff force of 60 g was employed to prevent tissue damage. Tail flick latency in response to the application of radiant heat applied to the tail using an Algesia Meter (IITC, Woodland Hills, Calif.) was used to assess acute nociception and its modulation by i.t. morphine as we described previously (See e.g., Liu, N.J. and A. R. Gintzler, Spinal Endomorphin 2 Antinociception and the Mechanisms That Produce It Are Both Sex-and Stage of Estrus Cycle-Dependent in Rats. J Pain, 2013. 14(11): p. 1522-1530). Intensity of the radiant heat was adjusted such that baseline values were in the range of 3.0-4.5 sec. Tissue damage was prevented by imposing a 10 sec cutoff. All testing was performed blind to treatment.

Drugs: In embodiments, a drug for use in accordance with the present disclosure includes caveolin-1 scaffolding domain peptide (c1-sd82-101) fused at the N-terminus to the cell-permeable antennapedia internalization sequence (43-58). An example of CSD suitable for use herein includes CSD (H-Arg-Gln-Ile-Lys-Ile-Trp-Phe-Gln-Asn-Arg-Arg-Met-Lys-Trp-Lys-Lys-Asp-Gly-Ile-Trp-Lys-Ala-Ser-Phe-Thr-Thr-Phe-Thr-Val-Thr-Lys-Tyr-Trp-Phe-Tyr-Arg-OH) (SEQ ID NO: 1). In embodiments, the first 16 amino acids of SEQ ID NO: 1 are characterized as an antennapedia internalization sequence, fused on the N-terminus of the scaffolding domain sequence (H-Asp-Gly-Ile-Trp-Lys-Ala-Ser-Phe-Thr-Thr-Phe-Thr-Val-Thr-Lys-Tyr-Trp-Phe-Tyr-Arg-OH) (SEQ ID NO: 2).

In another embodiment, a scrambled caveolin-1 scaffolding domain peptide (C1-SD₈₂₋₁₀₁) fused at the N-terminus to the antennapedia internalization sequence (43-58) may be used in the examples to serve as a useful negative control for studies using Caveolin-1 Scaffolding Domain Peptide. A non-limiting example of a S-CSD having the following amino acid sequence: (H-Arg-Gln-Ile-Lys-Ile-Trp-Phe-Gln-Asn-Arg-Arg-Met-Lys-Trp-Lys-Lys-Trp-Gly-Ille-Asp-Lys-Ala-Ser-Phe-Thr-Thr-Phe-Thr-Val-Thr-Lys-Tyr-Trp-Phe-Arg-Tyr-OH) (SEQ ID NO: 3)

In embodiments, CSD and S-CSD were obtained from Millipore Corporation (Bedford, Mass.). 3-(4-chlorophenyl) 1-(1,1-dimethylethyl)-1H-pyrazolo[3,4-d]pyrimidin-4-amine (PP2, a Src kinase inhibitor) and 1-Phenyl-1H-pyrazolo[3,4-d]pyrimidin-4-amine (PP3, a negative control for PP2) was obtained from Tocris (Ellisville, Mo.). Morphine sulfate and 75 mg morphine base pellets were obtained from NIDA. CSD (up to 10 μg), S-CSD (10 μg), PP2 (300 ng) and PP3 (300 ng) were solubilized in dimethyl sulfoxide (DMSO, up to 5 μl) while morphine sulfate (up to 20 μg) was prepared in 5 μl saline.

### Morphine Dosages Used to Generate Morphine Tolerance in Rat

The dose of morphine used to generate morphine tolerance: spinally: intrathecal 15 μg daily ×6 days; systemically: s.c. 75 mg morphine based/pellet, 6 pellets in 6 days. It is noted that the systemic dose is more than 1000× the intrathecal dose.

### Data Analysis

Only-way ANOVA was used to compare treatment effect within group and two-way repeated measure ANOVA was used to compare treatment by time/dose effects among groups. Bonferroni post hoc test identified specific groups between which significant effects were observed. P<0.05 was considered significant. Statistical comparisons were made using Prism 6 software. Data are expressed as the mean+/−standard error of the mean (SEM).

### Results

Combined Intrathecal (I.t.) Treatment with CSD and PP2 Prevents Development of Spinal Opioid Antiallodynic Tolerance Induced by Chronic i.t. Morphine.

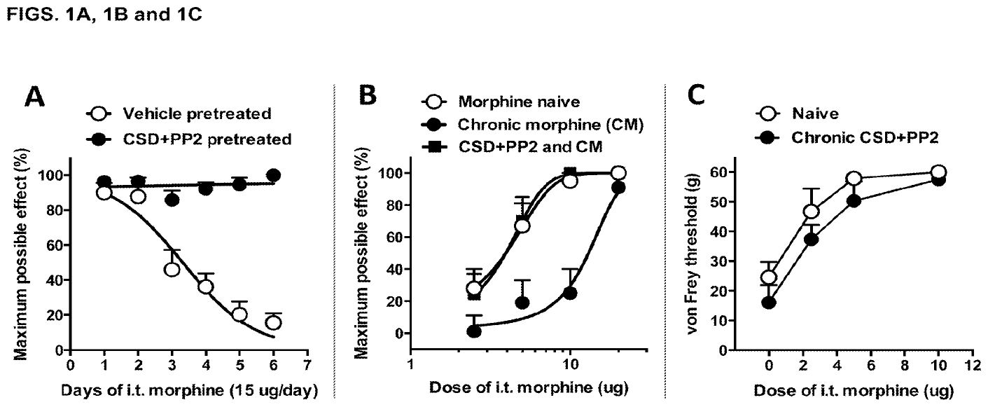

In these experiments, spinal opioid analgesic tolerance was achieved by the i.t. application of 15 μg morphine, once daily for six days. Referring to FIG. 1A, a graph depicts an opened circle (control group) that illustrates the complete loss of antiallodynic responsiveness to i.t. morphine (i.e., tolerance) following daily i.t. application of 15 μg morphine for 6 days, n=6. I.t. CSD+PP2 (10 μg and 300 ng, respectively) pre-treatment (45 min before i.t. morphine) completely prevented tolerance development (filled circles; n=10). Vehicle (DMSO) for CSD+PP2, which was included in the control group had no effect on tolerance development. More specifically, FIG. 1A depicts the spinal treatment of the present disclosure produced substantial tolerance, essentially eliminating antiallodynic responsiveness to i.t. morphine (15 μg), assessed using the electronic von Frey test. By day 3, roughly half the initial antiallodynic effect of i.t morphine was no longer manifest and by day 6, essentially the totality of i.t. morphine responsiveness had been eliminated. In striking contrast, there was no loss of morphine's effectiveness in chronic morphine-treated animals that had been pretreated intrathecally with CSD+PP2 (10 μg and 300 ng, respectively), 45 min prior to i.t. morphine. Two-way ANOVA revealed a significant treatment effect (i.t. CSD+PP2 vs. vehicle; n=10 and 6, respectively) (F_{1.14}=92.3, p<0.0001) after day 2 of i.t. morphine (p<0.0001 for days 3-6).

Referring now to FIG. 1B, a graph depicts dose-responsiveness to i.t. morphine determined in morphine naive rats (opened circle; n=7), morphine tolerant rats (filled circle; n=6), and rats that had received i.t. CSD+PP2 45 min prior to chronic daily i.t. morphine (CM) (filled square; n=10). Morphine tolerant rats required 3.4 times more morphine than naive rats to elicit comparable antiallodynia (ED₅₀=13.1 μg vs. 3.9 μg). In contrast, i.t. morphine was essentially equi-effective in CSD+PP2+CM group vs. opioid naïve rats, i.e., CSD+PP2 eliminated morphine antiallodynic tolerance. More specifically, FIG. 1B depicts that the totality of the rightward shift of i.t. morphine dose-responsiveness (2.5-20 μg) was eliminated by pretreatment with CSD+PP2, revealing that their tolerance abating effect was not limited by the dose of i.t. morphine. Two-way ANOVA showed a significant treatment effect among the 3 groups: F2.15=54.11, p<0.0001). Significant differences were observed between chronic morphine (CM) group (n=5) and CSD+PP2+CM group (n=6) as well as between CM group and morphine naïve group (n=7) (p<0.0001 for both comparisons), but not between CSD+PP2+CM and opioid naïve animals (p>0.05). The latter comparison indicates that pretreatment with i.t. CSD+PP2 abolished development of spinal morphine antiallodynic tolerance. This effect of i.t. CSD+PP2 results specifically from its anti-tolerance action since (a) i.t treatment with vehicle (DMSO) had no effect on tolerance development and (b) as shown in FIG. 1C, a graph depicts chronic i.t. treatment with CSD+PP2 did not alter the antiallodynic effect of acute i.t. morphine in the absence of chronic spinal morphine treatment (F_{1.18}=3.4, p>0.05, n=4 for each point), i.e., i.t. CSD+PP2 neither directly influenced basal von Frey thresholds nor acute spinal morphine antiallodynic responsiveness. Instead i.t. CSD+PP2 specifically interrupted mechanisms underlying opioid tolerance development, enabling i.t. morphine to produce normative antiallodynia despite the chronic morphine treatment. More specifically, FIG. 1C depicts chronic i.t. CSD+PP2 daily for 6 days did not significantly affect either basal von Frey thresholds or i.t. morphine dose responsiveness, in the absence of chronic i.t. morphine treatment. n=4 for each point. CSD (caveolin-1 scaffolding domain peptide); PP2 (selective c-Src inhibitor); CM (chronic morphine).

I.t. Treatment with CSD+PP2 Reverses Spinal Opioid Antiallodynic Tolerance.

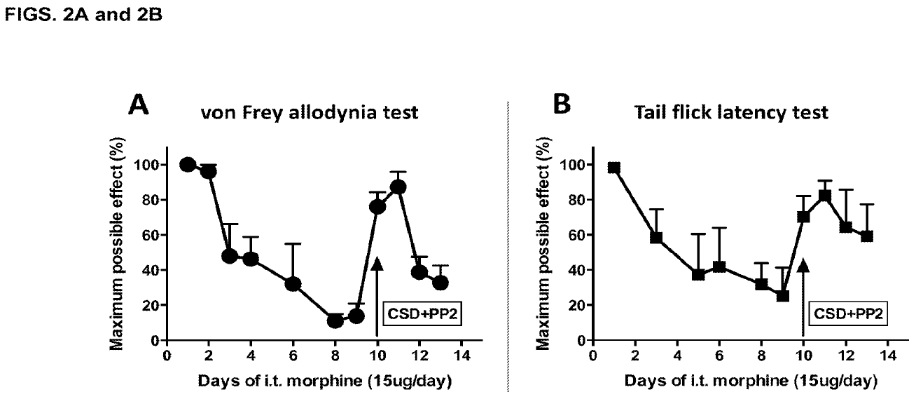

Prevention of the development of spinal opioid antiallodynic tolerance by i.t. CSD+PP2 does not necessarily indicate the ability of this treatment to reverse tolerance, once it has fully developed. In order to determine if i.t. CSD+PP2 was also able to acutely reverse opioid antiallodynic tolerance, the effect of intrathecally applying CSD+PP2 on i.t. morphine antiallodynia was determined subsequent to tolerance development (FIG. 2A). For example, FIG. 2A provides a von Frey allodynia test showing that i.t. morphine had lost most of its antiallodynic effectiveness after 9 days of daily i.t. morphine treatment (15 μg). I.t. CSD+PP2 (10 μg and 300 ng, respectively) applied on day 10, 45 min before i.t. morphine, fully restored spinal antiallodynic morphine responsiveness, which lasted for 2 days (n=8). One-way ANOVA revealed a significant treatment effect among days 1, 9, and 11 (F_{2.21}=58.4, p<0.0001), significant differences being observed between effects on day 1 vs. day 9 (when antiallodynic tolerance was fully manifest) (P<0.001) and effects on day 9 vs. day 11 (when i.t. CSD+PP2 resulted in the biggest reversal) (p<0.01), but not between days 1 and 11 (p>0.05). The latter comparison revealed that tolerance reversal was complete. As illustrated in FIG. 2A (n=8), following 9-days of spinal morphine treatment, morphine tolerance was fully manifest, essentially eliminating the totality of morphine's antiallodynic effect. The i.t. application of CSD+PP2 on day 10 (45 min prior to intrathecally administering morphine) restored >95% of the initial i.t morphine-induced antiallodynia.

Referring now to FIG. 2B, a tail flick latency test was also used to assess i.t. morphine analgesic tolerance. As is illustrated in FIG. 2A, i.t. morphine lost most of its analgesic effect 9 days following daily 15 μg i.t. morphine treatment. I.t. CSD+PP2, applied on day 10, 45 min before i.t. morphine, fully restored morphine antinociception, which lasted for 4 days (n=5). CSD (caveolin-1 scaffolding domain peptide); PP2 (selective c-Src inhibitor).

The effect of i.t. CSD+PP2 on tail flick latency was also investigated (FIG. 2B) in order to investigate whether or not the acute anti-tolerance effect of i.t. CSD+PP2 was pain modality specific. As was observed for neuropathetic pain, one-way ANOVA revealed a significant treatment effect (F_{2.11}=17.5, p<0.001; n=5) among days 1, 9, and 11, significant differences being observed on day 1 vs. day 9 (when tolerance was fully manifest) (P<0.001) and on day 9 vs. 11 (when CSD+PP2 showed the biggest reversal) (p<0.01). No treatment effect was observed between day 1 and day 11 (p>0.05), indicating that tolerance reversal was essentially complete, i.t. CSD+PP2 restoring >90% of the analgesic effect of i.t. morphine manifest prior to chronic spinal morphine.

I.t. CSD or PP2, Each Administered Individually, Reverses Spinal Opioid Antiallodynic Tolerance Induced by Chronic i.t. Morphine.

Referring to FIGS. 3A and 3B, it is shown that i.t treatment with either CSD or PP2 reverses i.t. morphine tolerance. Spinal antiallodynic tolerance to i.t. morphine was generated as described in the description of FIG. 1 above. The von Frey allodynia test revealed that i.t. morphine had lost most of its antiallodynic effect 7-8 days following daily i.t. 15 μg morphine. CSD (10 μg) (FIG. 3A) or PP2 (300 ng) (FIG. 3B) was intrathecally applied 45 min before i.t. morphine on day 8 or 9 of chronic morphine treatment when maximal morphine tolerance was manifest. I.t. CSD fully restored i.t morphine antiallodynia, whereas i.t. PP2 restored approximately 60% of opioid naïve i.t. morphine responsiveness. The reversal of morphine tolerance by either CSD or PP2 lasted for multiple days. n=6 and 5 for CSD and PP2, respectively. CSD (caveolin-1 scaffolding domain peptide); PP2 (selective c-Src inhibitor).

More specifically, and without wishing to be bound by this disclosure it was thought that CSD and PP2 would interfere with tolerance by acting at two sequentially interconnected components of tolerance mechanisms, CSD interfering with scaffolding by competing with the scaffolding domain within Cav1 while PP2 inhibits Src kinase, known to phosphorylate Tyr 114 of Cav1, thereby modulating its scaffolding ability. Accordingly, the individual i.t. application of either CSD (10 μg; FIG. 3A) or PP2 (300 ng; FIG. 3B) was tested to see if it would be sufficient to interrupt opioid antiallodynic tolerance. For the CSD treated group (n=6), one-way ANOVA showed a significant treatment effect (F2.15=14.5, p<0.001) between day 1 and day 7 (when tolerance was fully manifest) (P<0.001) as well as between day 7 and day 8 (when CSD showed the biggest reversal) (p<0.01), but not between day 1 and day 8 (p>0.05). The latter comparison reveals that tolerance reversal was complete.

The PP2 treated group followed suit. One-way ANOVA showed a significant treatment effect (F_{2.1}2=14.3, p<0.001) between day 1 and day 8 (when tolerance was fully manifest) (P<0.001) as well as between day 8 and day 9 (when PP2 produced the biggest reversal) (p<0.05) (n=5). As is shown in FIGS. 3A and 3B, by day 7 of spinal morphine treatment, tolerance had developed to i.t. morphine, essentially eliminating the totality of its antiallodynic effects. On day 9, i.t. PP2 (FIG. 3B), applied intrathecally 45 min prior to the i.t. morphine, attenuated spinal opioid antiallodynic tolerance, substantially restoring the ability of i.t. morphine to elevate allodynic (von Frey) thresholds.

I.t. CSD Blocks the Development of Opioid Antiallodynic as Well as Antinociceptive Tolerance Induced by Chronic Systemic Morphine.

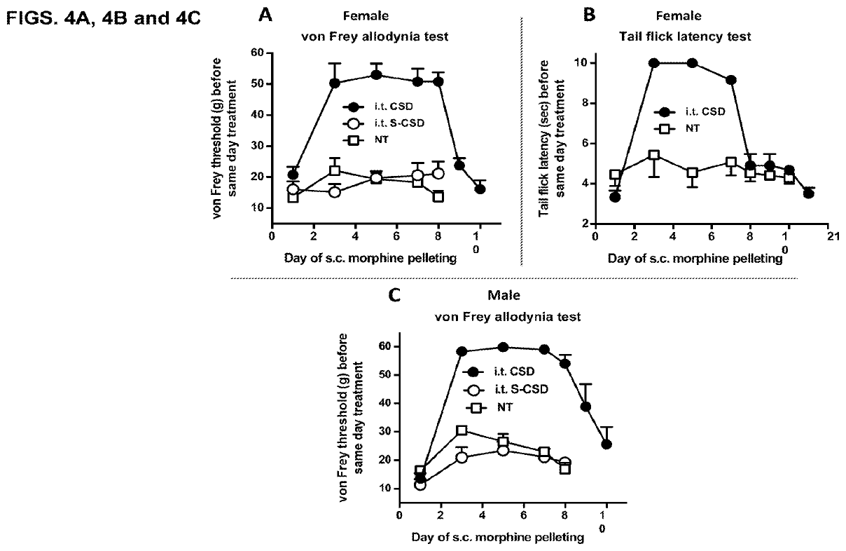

Referring now to FIGS. 4A, 4B and 4C, intrathecal CSD prolongs systemic morphine antiallodynia and antinociception and prevents antiallodynic/antinociceptive tolerance development to systemic morphine in female as well as in male rats. Referring to FIG. 4A, von Frey thresholds of the rear paw ipsilateral to spinal nerve ligation were quantified immediately prior to the same day intrathecal treatment and subcutaneous implantation of continuous long-lasting release morphine pellets (performed on days 1, 3, and 5). Values reflect effect from on-boarded morphine from the previous day's subcutaneous morphine pelleting. CSD or S-CSD, 6 μg each, was intrathecally applied 45 min before subcutaneous morphine pellet implantation. Day 1 represents opioid naïve basal von Frey thresholds. Throughout the ensuing days, the antiallodynic effects of the on-boarded morphine continued to be robustly manifest in rats that had received intrathecal CSD. This was reflected by the increase to cutoff in von Frey thresholds, which persisted for 3-days following the last intrathecal CSD treatment (on day 5). In contrast, the antiallodynia resulting from the subcutaneous morphine pellets remained minimal in rats that had received intrathecal S-CSD (open circle) or no intrathecal treatment (open square), reflecting antiallodynic morphine tolerance. n=8, 6, and 12, for CSD, S-CSD and NT group, respectively. Referring to FIG. 4B, the tail flick latency test was used to assess antinociception of on-boarded systemic morphine and antinociceptive tolerance development to systemic morphine in 3 of the intrathecal CSD-treated/subcutaneous morphine-pelleted rats used in FIG. 4A, as well as 3 subcutaneous morphine pelleted rats that did not get intrathecal treatment. As in FIG. 4A, day 1 represents opioid naïve basal tail flick latency, and subsequent tail flick tests were performed immediately prior to same day treatment. Also, as in FIG. 4A, values reflect effect of on-boarded morphine from the previous day's subcutaneous morphine pelleting. Intrathecal CSD prevents morphine tolerance development, reflected by the close to cutoff tail flick latency, which persisted for 2-days following the last intrathecal CSD treatment (on day 5). Referring to FIG. 4C, intrathecal CSD works identical in males as in females (FIG. 4A). n=7, 7 and 6, for CSD, S-CSD and NT group, respectively. CSD (caveolin-1 scaffolding domain competing peptide); S-CSD (scrambled caveolin-1 scaffolding domain competing peptide); i.t. (intrathecal); NT (no treatment); s.c. (subcutaneous).

More specifically, recognizing that opioids are frequently given systemically, the ability of i.t. CSD to prevent opioid antiallodynic tolerance induced by systemic chronic morphine, administered via the s.c. implantation of morphine base pellets, was investigated in both females and males. The data obtained in females are shown in FIG. 4A that included 3 groups: animals receiving s.c. morphine pellets (n=12), morphine-pelleted animals receiving i.t. CSD pretreatment, administered 45 min before morphine pellet implantation on days 1, 3 and 5 (n=8) and morphine-pelleted animals receiving i.t. pretreatment with scrambled CSD (S-CSD, administered as described for i.t. CSD) (n=6). Two-way ANOVA showed a significant treatment effect among the 3 groups (F_{2.23}=57.70, p<0.0001), significant differences being observed between the CSD and S-CSD pretreated groups (p<0.0001) as well as between chronic morphine pelleted rats with vs. without i.t. CSD pretreatment (p<0.0001). Two-days after s.c. implantation of morphine base pellets in the absence of i.t. CSD, the antiallodynic effect of the systemic morphine was minimal relative to a cutoff morphine effect before pelleting (data not shown). Notably, in the presence of i.t. CSD (10 μg), the antiallodynia produced by the implanted morphine pellets was fully manifest, as reflected by the von Frey thresholds reaching cutoff. Blockade of opioid antiallodynic tolerance development persisted through day 8, 3 days after the last i.t. application of CSD. On day 9 (4 days after the last i.t. application of CSD), when spinal CSD concentrations presumably started to decline, the manifestation of opioid antiallodynic tolerance began to emerge, indicating that blockade of tolerance development by CSD was not an artifact of the random occurrence of tolerance resistance in this particular group of rats. In contrast to CSD, i.t. pretreatment with S-CSD (10 μg applied 45 min before i.t. morphine) failed to alter morphine tolerance development, i.e., there was no difference in tolerance development to chronic systemic morphine between animals that received i.t. S-CSD vs. no spinal treatment.

Similar to investigating the ability of i.t. CSD to prevent opioid antiallodynic tolerance induced by systemic chronic morphine, the ability of i.t. CSD to block systemic opioid analgesic tolerance was also investigated. These data were shown in FIG. 4B. FIG. 4B include 2 groups of rats, i.t. CSD group vs. no i.t. treatment group (n=3 each). Both groups of rats are from the corresponding groups in FIG. 4A, i.e., the 3 pairs of rats not only got von Frey test, but also got tail flick latency test to check morphine analgesic effect. Two-way ANOVA showed a significant treatment effect between the 2 groups (F_{1.4}=37.03, p<0.01). In the presence of i.t. CSD (10 μg), the antinociception produced by the implanted morphine pellets was fully manifest, as reflected by the tail flick latency reaching cutoff. Blockade of opioid antinociceptive tolerance development persisted through day 7, 2 days after the last i.t. application of CSD. In contrast to i.t. CSD, no i.t. treatment group did not show any significant morphine effect, i.e., morphine antinociceptive tolerance persisted with no alteration.

Data obtained in males is shown in FIG. 4C, which also included 3 groups: animals receiving s.c. morphine pellets (n=6), morphine-pelleted animals receiving i.t. CSD pretreatment, administered 45 min before morphine pellet implantation on days 1, 3 and 5 (n=7) and morphine-pelleted animals receiving i.t. pretreatment with scrambled CSD (S-CSD, administered as described for i.t. CSD) (n=7). Two-way ANOVA showed a significant treatment effect among the 3 groups (F_{2.51}=153.4, p<0.0001), significant differences being observed between the CSD and S-CSD pretreated groups (p<0.001) as well as between chronic morphine pelleted rats with vs. without i.t. CSD pretreatment p (p<0.0001). Two-days after s.c. implantation of morphine base pellets in the absence of i.t. CSD, the antiallodynic effect of the systemic morphine was minimal relative to a cutoff morphine effect before pelleting (data not shown). Notably, in the presence of i.t. CSD (10 μg), the antiallodynia produced by the implanted morphine pellets was fully manifest, as reflected by the von Frey thresholds reaching cutoff. Blockade of opioid antiallodynic tolerance development persisted through day 8, 3 days after the last i.t. application of CSD. On day 9 (4 days after the last i.t. application of CSD), when spinal CSD concentrations presumably started to decline, the manifestation of opioid antiallodynic tolerance began to emerge, indicating that blockade of tolerance development by CSD was not an artifact of the random occurrence of tolerance resistance in this particular group of rats. In contrast to CSD, i.t. pretreatment with S-CSD (10 μg applied 45 min before i.t. morphine) failed to alter morphine tolerance development, i.e., there was no difference in tolerance development to chronic systemic morphine between animals that received i.t. S-CSD vs. no spinal treatment.

I.t. CSD+PP2 Restores i.t. Morphine Dose Responsiveness in Animals Treated Chronically with Systemic Morphine.

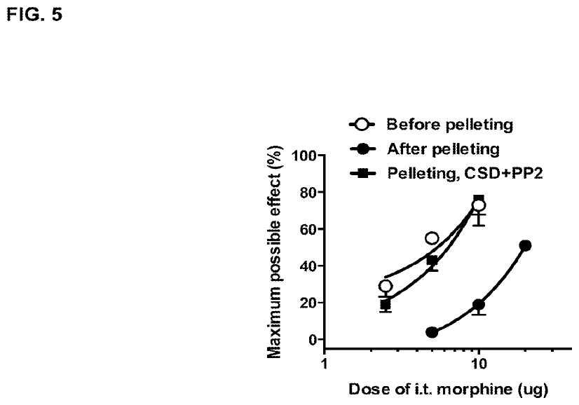

Referring now to FIG. 5, i.t. CSD and PP2 reverse spinal tolerance to systemic morphine is shown. Dose-responsiveness to i.t. morphine in opioid naive rats (opened circle; n=8), rats receiving chronic systemic morphine via s.c. implanted morphine pellets (filled circle; n=8) and rats receiving chronic systemic morphine along with a one-time i.t. application of CSD+PP2 (10 μg and 300 ng, respectively; administered 45 min prior to assessing i.t. morphine dose-responsiveness) (filled square; n=8). As expected, the i.t. morphine ED₅₀ was 3.6-fold greater in chronic systemic morphine-treated (tolerant) rats than opioid naive rats (19.7 μg vs. 5.5 μg), reflecting antiallodynic tolerance development. In contrast, the i.t. morphine ED₅₀ observed in chronic morphine-treated rats that received i.t. CSD+PP2 prior to dose-response quantification was essentially the same as that of opioid naive rats (6.4 μg vs. 5.5 μg), underscoring the ability of i.t. CSD+PP2 to acutely eliminate systemic morphine tolerance, even after its full development. CSD (caveolin-1 scaffolding domain peptide); PP2 (selective Src inhibitor).

More specifically, in order to assess the full extent of the ability of i.t. CSD+PP2 to reverse tolerance to spinal morphine's antiallodynic actions in animals receiving chronic systemic morphine, the ability of i.t. CSD+PP2 to restore spinal morphine antiallodynic dose responsiveness was investigated (FIG. 5). Groups consisted of opioid naïve animals (‘before pelleting’), opioid tolerant animals (‘after pelleting’) and morphine pelleted animals that also received a one-time i.t. application of CSD+PP2) (‘pelleting, CSD+PP2’), n=8 for each group. Two-way ANOVA revealed significant treatment effects among the groups (F_{2.47}=194.0, p<0.0001). As expected, i.t. morphine dose responsiveness obtained in opioid naïve animals was significantly different from i.t. dose responsiveness obtained in the morphine pelleted animals (p<0.01). Strikingly, i.t. morphine antiallodynic dose responsiveness obtained in morphine pelleted (opioid tolerant) animals also significantly differed from that obtained in morphine pelleted animals that had received a one-time i.t. application of CSD+PP2, 45 min prior to quantifying i.t. morphine dose responsiveness (p<0.001). Moreover, morphine dose responsiveness obtained in the latter group did not significantly differ from that obtained in opioid naïve animals (p>0.05), indicating that i.t. CSD+PP2 restored normative spinal morphine antiallodynia. These data are shown in FIG. 5, which illustrates that chronic systemic morphine shifted i.t. morphine ED₅₀ from 5.5 μg to 19.7 μg. The totality of this rightward shift in morphine dose responsiveness (antiallodynic tolerance) was obliterated following the i.t. 45 min pretreatment with CSD+PP2 (10 μg and 300 ng, respectively), i.e., i.t. CSD+PP2 pretreatment of morphine tolerant animals reinstated spinal morphine antiallodynic responsiveness (ED₅₀=6.4 μg), which was indistinguishable from that observed in opioid naïve rats (5.5 μg).

I.t. CSD or PP2 Reverses Spinal Opioid Tolerance Induced by Chronic Systemic Morphine.

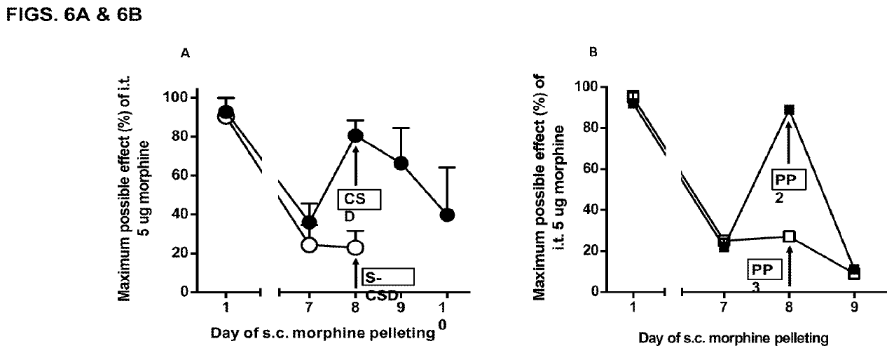

Referring now to FIGS. 6A and 6B, graphs depicts intrathecal CSD or PP2 reverses spinal tolerance induced by systemic chronic morphine. Experiments were performed as described above in the description of FIG. 5. Intrathecal CSD or PP2 (10 microgram, 300 ng, respectively; applied on day 8, 45 min before i.t. morphine) essentially fully restored i.t. morphine antiallodynic action. This effect lasted more than 24 hours for CSD but less than 24 hours for PP2. In contrast, i.t. S-CSD (10 microgram) or PP3 (300 ng), a negative control for CSD and PP2, respectively, applied 45 min before i.t. morphine, failed to alter antiallodynic action of i.t. morphine. These results indicate that i.t. CSD or PP2 can reverse spinal morphine antiallodynic tolerance induced by chronic systemic morphine. CSD (caveolin-1 scaffolding domain competing peptide); S-CSD (scrambled caveolin-1 scaffolding domain competing peptide); PP2 (selective c-Src inhibitor); PP3 (negative control for PP2).

More specifically, spinal treatment with CSD was effective in reversing systemic morphine-induced spinal opioid tolerance, once it had been fully developed (FIG. 6A). One-way ANOVA showed a significant treatment effect (F_{2.24}=6.03, p<0.01, n=8) between day 1 and day 7 (when tolerance was fully manifest) (P<0.01) as well as between day 7 and day 8 (indicating acute tolerance reversal by CSD) (p<0.05), but not between day 1 and day 8 (p>0.05). The latter comparison indicating that tolerance had been acutely eliminated. FIG. 6A illustrates that chronic systemic morphine had lost virtually all of its antiallodynic effect seven days following the onset of pelleting. Forty-five min after 10 μg i.t. CSD, spinal morphine antiallodynic tolerance was essentially fully mitigated, i.t. morphine producing a robust antiallodynia that was comparable to that produced prior to the onset of morphine pelleting, i.e., i.t. CSD fully restored i.t. morphine antiallodynic action. Full reversal of tolerance development was also effectuated after i.t. application of lower dose of CSD (3, 1, or 0.33 μg; data not shown). The opioid tolerance-reversing effect of i.t. CSD persisted in excess of 24 hours. In contrast, i.t. S-CSD had no effect on the chronic systemic morphine-induced tolerance (p>0.05 compare to day 7, n=6).

As was observed for CSD, i.t. PP2 (300 ng applied on day 8, 45 min before i.t. morphine), also fully restored i.t. morphine antiallodynic effects, which had been diminished by approximately 80% (FIG. 6B). One-way ANOVA showed a significant treatment effect (F_{2.18}=301.7, p<0.0001). The significant effect was between day 1 and day 7 (when tolerance fully manifested) (P<0.0001) as well as between day 7 and day 8 (when CSD showed biggest reversal) (p<0.0001). In contrast, a significant effect was not observed between day 1 and day 8 (p>0.05; n=7), indicating that i.t. PP2 fully reversed spinal morphine tolerance. Interestingly, on day 9, one day after i.t. PP2, opioid antiallodynic tolerance was once again manifest, indicating that the reversal observed on day 8 did not result from a random, sudden loss of tolerance, confounding interpretation. Importantly, the negative control for PP2, PP3, was devoid of any effect on opioid antiallodynic tolerance. One-way ANOVA of the PP3 group showed a significant treatment effect (F_{2.6}=185.6, p<0.0001), reflecting spinal morphine antiallodynic tolerance. The significant effect was between day 1 and day 7 (when tolerance was fully manifested) (P<0.0001) as well as between day 1 and day 8 (P<0.0001), illustrating the persistence of this tolerance after i.t. PP3, but not between day 7 and day 8 (p>0.05). The latter underscores the inability of i.t. PP3 to reverse tolerance. n=3.

An Absence of Desensitization to the Opioid Tolerance Mitigating Effect of I.t. CSD Following a Reapplication.

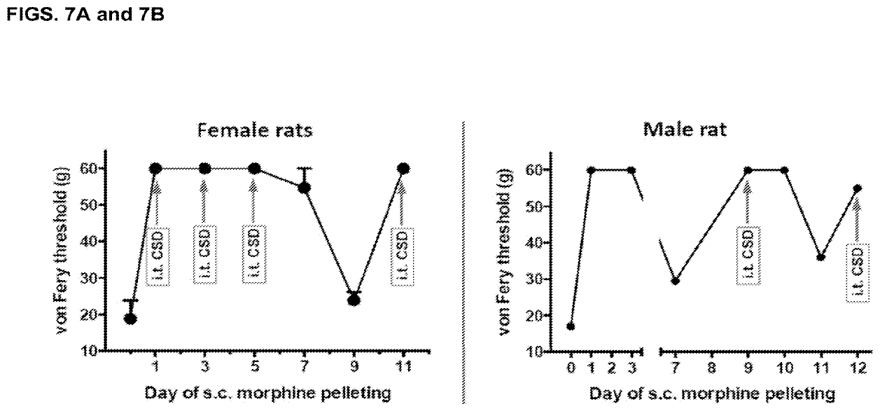

Referring now to FIGS. 7A and 7B, graphs depicting von Frey thresholds are provided. Here, rats were treated and tested as described in previous figures. In brief, von Frey thresholds (g) shown were determined before morphine pelleting (day 0) as well as 30 min after morphine pelleting (with or without an intrathecal (i.t.) CSD 45 min pretreatment). Left panel: data from female rats show that pretreatment with i.t. CSD (6 μg) before morphine pelleting prevented morphine tolerance development as indicated by cutoff von Frey thresholds. On day 9 of morphine pellet exposure, 4 days after the last injection of i.t. CSD on day 5 of morphine pelleting, morphine antiallodynia became minimal, indicating morphine antiallodynic tolerance development. Notably, however, when i.t. CSD was re-applied on day 11, morphine antiallodynia was fully manifest, indicating that desensitization did not develop to the opioid tolerance mitigating effects of i.t. CSD following a reapplication. Right pane: data from a male rat shows that on day 7, two days after the last morphine pelleting, morphine antiallodynic tolerance substantially eliminated i.t. morphine antiallodynia. This tolerance was reversed by i.t. 6 μg CSD on day 9, which lasted through day 10. Moreover, additional reapplication of i.t. CSD on day 12 again restored morphine antiallodynia, again indicating the absence of desensitization to the opioid tolerance mitigating effect of i.t. CSD.

### Discussion

The development of opioid analgesic/antiallodynic tolerance results in the requirement for profound dose escalation. Since this exacerbates side effects and addiction liability, the need for dose escalation has long been a major confound in the clinical utilization of opioids for managing nociception. Current findings reveal that the scaffolding protein Cav1 and the nonreceptor tyrosine kinase Src (which phosphorylates Cav1, thereby altering its scaffolding properties) are two likely targets for developing novel adjunctive pharmacotherapies to mitigate, if not abolish, opioid analgesic/antiallodynic tolerance.

The relevance of protein scaffolding to the development of opioid tolerance was predicated on multiple factors. These include (a) the presence of MOR (and the kappa-opioid receptor) within multimeric signaling complexes (See e.g., Liu, N.J., et al., Estrogens synthesized and acting within a spinal oligomer suppress spinal endomorphin 2 antinociception: ebb and flow over the rat reproductive cycle. Pain, 2017.158(10): p. 1903-1914 and Liu, N.J., et al., Plasticity of signaling by spinal estrogen receptor alpha, kappa-opioid receptor and mGluRs over the rat reproductive cycle regulates spinal endomorphin 2 antinociception: relevance of endogenous biased agonism. J Neurosci, 2017. 37: p. 11181-11191), which invariably require a structural backbone for their formation; (b) the ability of chronic morphine to induce coordinated, interrelated changes in a wide spectrum of signaling molecules downstream from MOR, which is likely to be facilitated by protein scaffolding; as would be (c) the increased emergence and prominence of new signaling strategies in response to chronic morphine, well documented to occur in response to chronic morphine (See e.g., Gintzler, A. R. and S. Chakrabarti, Opioid tolerance and the emergence of new opioid receptor-coupled signaling. Molecular Neurobiology, 2000. 21: p. 21-33, and Gintzler, A. R. and S. Chakrabarti, The ambiguities of opioid tolerance mechanisms: barriers to pain therapeutics or new pain therapeutic possibilities. J Pharmacol Exp Ther, 2008. 325(3): p. 709-13.

The combined i.t. application of CSD (which, competes with the scaffolding domain of Cav1 for binding to membrane proteins thereby effectively blocking Cav1 scaffolding and signaling) and PP2 (which inhibits Src phosphorylation of Cav1) eliminated the development of spinal tolerance to the antiallodynic effect of intrathecally-applied morphine. Importantly, i.t. CSD+PP2 abolished tolerance development irrespective of the route of chronic morphine administration, abolishing tolerance induced by chronic morphine applied spinally (via daily i.t. application) as well as systemically (via s.c. implantation of morphine base pellets). Since in our formulation, Src is posited to act upstream of Cav1, modifying its scaffolding functionality via phosphorylation, it was anticipated that the individual i.t. application of CSD (i.e., interrupting Cav1 scaffolding) or PP2 (inhibiting Cav1 phosphorylation) would mitigate tolerance development to the same extent as would applying CSD together with PP2. As expected, i.t. CSD, in the absence of PP2, or PP2 (in the absence of CSD) retained their ability to eliminate spinal morphine antiallodynic tolerance. This underscores the putative clinical utility of interfering with Cav1 scaffolding as an adjunctive pharmacotherapy accompanying the use of opioids for pain management.

Many of the proposed biochemical underpinnings of opioid tolerance are not consonant with acute reversal of tolerance once it has fully developed. For example, the long-held view that MOR internalization/downregulation and adenylyl cyclase super activation are foundational to opioid analgesic tolerance (See e.g., Pineyro, G. and E. Archer-Lahlou, Ligand-specific receptor states: implications for opiate receptor signalling and regulation. Cell Signal, 2007. 19(1): p. 8-19; Law, P. Y., D. S. Hom, and H. H. Loh, Loss of opiate receptor activity in neuroblastoma X glioma NG108-15 hybrid cells after chronic opiate treatment. A multiple-step process. Mol Pharmacol, 1982. 22(1): p. 1-4; Sharma, S. K., W. A. Klee, and M. Nirenberg, Opiate-dependent modulation of adenylate cyclase. Proc. Natl. Acad. Sci. USA, 1977. 74(8): p. 3365-3369) would not be expected to be readily reversed within min following pharmacological intervention, thereby restoring naïve levels of antiallodynic responsiveness to i.t. morphine. Strikingly, this is exactly what was observed following i.t. CSD+PP2 or their individual i.t. application. In fact, i.t. CSD (0.33 μg) unmasked robust morphine (from on boarded morphine pellet) antiallodynia in systemic morphine tolerant animals, even in the absence of virtually any pretreatment with i.t. morphine. Of note, the acute reversal of morphine antiallodynic tolerance by CSD (but not S-CSD) and/or PP2 (but not PP3) was not permanent. Within 24-72 hours, tolerance was once again observed. This reinstatement of tolerance presumably resulted from the run-down of CSD or PP2 concentrations, resulting in the reinstatement of Cav1 scaffolding functionality, characteristic of the opioid tolerant condition. The acute reversal and reestablishment of morphine antiallodynic tolerance reveals a fluid, temporally dynamic dimension of opioid tolerance, not explicitly provided for in current models of tolerance. Furthermore, reapplication of CSD, after its effect has dissipated, once again quickly reverse antiallodynic tolerance indicating that tolerance does not develop to the tolerance-abating effects of CSD.

The magnitude of acute reversal by i.t. CSD and/or PP2 of spinal morphine antiallodynic tolerance as reflected by unmasking the antiallodynic effects of on boarded morphine pellets was quite variable. This contrasts with the magnitude of the acute reversal by i.t. CSD and/or PP2 of tolerance to the antiallodynic effects of intrathecally-applied morphine, which is consistently robust. This dichotomy likely results from the spinally restricted loci of action of intrathecally applied CSD and/or PP2 since such localization would more consistently impact tolerance to the antiallodynia mediated by spinal opioid receptors (vis a vis i.t. morphine) than the antiallodynia mediated by opioid receptors located supraspinally as well as spinally, as occurs with systemic morphine. The ability of CSD and/or PP2 to prevent and/or reverse supraspinal morphine antiallodynic tolerance when they are applied to supraspinal sites remains to be determined.

The variable ability of i.t. CSD/PP2 to unmask the antiallodynic effects of subcutaneously implanted morphine pellets also contrasts with our observation that the identical i.t. treatment prior to implanting morphine base pellets invariably and robustly maintained the antiallodynic effects of implanted morphine pellets (i.e., prevented tolerance development) such that the von Frey cutoff threshold was routinely manifest. This dichotomy can result from the bidirectional communication between the spinal cord and brain, e.g., altered spinal cord excitability alters ascending messages, which in turn influences descending modulation arising from the midbrain (e.g., periaqueductal gray) and brainstem (e.g., rostroventromedial medulla) (See e.g., Bannister, K. and A. H. Dickenson, What the brain tells the spinal cord. Pain, 2016. 157(10): p. 2148-51; Bannister, K. and A. H. Dickenson, What do monoamines do in pain modulation? Curr Opin Support Palliat Care, 2016. 10(2): p. 143-8). A major difference between preventing vs. acutely reversing spinal opioid tolerance that results from chronic systemic morphine is that spinal tolerance prevention would obviate any influence of spinal opioid tolerance on ascending spinal brain communication (e.g., attenuating endogenous opioid modulation of ascending spinal nociceptive pathways) and resulting supraspinal adaptations, whereas acutely reversing spinal tolerance subsequent to its development would not. Moreover, the temporal profile for the offset of these adaptations might not coincide with that of the acute reversal of spinal opioid tolerance (and presumably the reinstatement of the unfettered activity of spinal ascending pathways). These considerations imply that while prevention and reversal of morphine antiallodynic tolerance by i.t. CSD/PP2 result from interrupting a common mechanism (Cav1 scaffolding), they likely do not share identical biochemical sequelae.

Multiple clinical reports indicate that persistent opioid exposure can, paradoxically, result in allodynia and hyperalgesia, which often differs from the original pain that is being treated (See e.g., Devulder, J., Hyperalgesia induced by high-dose intrathecal sufentanil in neuropathic pain. J Neurosurg Anesthesiol, 1997. 9(2): p. 146-8; De Conno, F., et al., Hyperalgesia and myoclonus with intrathecal infusion of high-dose morphine. Pain, 1991. 47(3): p. 337-9; Ali, N. M., Hyperalgesic response in a patient receiving high concentrations of spinal morphine. Anesthesiology, 1986. 65(4): p. 449: and Amer, S., N. Rawal, and L. L. Gustafsson, Clinical experience of long-term treatment with epidural and intrathecal opioids-a nationwide survey. Acta Anaesthesiol Scand, 1988. 32(3): p. 253-9). Analogous observations have also been reported in laboratory animals (See e.g., Mayer, D. J., J. Mao, and D. D. Price, The association of neuropathic pain, morphine tolerance and dependence, and the translocation of protein kinase C. NIDA Res Monogr, 1995. 147: p. 269-98). Interestingly, supraspinal adaptations to chronic systemic morphine (e.g., in the rostroventromedial medulla (RVM) and dorsolateral funiculus) has been shown to mediate chronic opioid-induced nociception (i.e., hyperalgesia, allodynia) as well as spinal morphine antinociceptive tolerance (See e.g., Vanderah, T. W., et al., Tonic descending facilitation from the rostral ventromedial medulla mediates opioid-induced abnormal pain and antinociceptive tolerance. J Neurosci, 2001. 21(1): p. 279-86). In fact, the former has been causally associated with the latter (See e.g,. Vanderah, T. W., et al., Tonic descending facilitation from the rostral ventromedial medulla mediates opioid-induced abnormal pain and antinociceptive tolerance. J Neurosci, 2001. 21(1): p. 279-86). This is noteworthy since i.t. CSD and/or PP2 similarly prevented/reversed spinal morphine antiallodynic tolerance produced by chronic morphine delivered either intrathecally or systemically, indicating that CSD and or PP2 interfere with a fundamental, shared component of multiple tolerance-producing adaptations. Additionally, these considerations suggest that CSD and/or PP2 could be effective in blocking/reversing chronic opioid-induced allodynia/hyperalgesia, further extending the clinical utility of opioids in chronic pain management.

Whereas CSD is specific for interrupting Cav1 scaffolding, Src has many substrates that could influence the development and maintenance of opioid tolerance. For example, chronic morphine-induced activation of Src can not only result in increased tyrosine phosphorylation of Cav1, altering its scaffolding properties, but also the tyrosine phosphorylation of MOR, (Kramer et al., 2000; Zhang, et al., 2009), which has been causally linked to the chronic morphine-induced activation of AC (AC super activation) as well as the conversion of MOR from a classical G-protein coupled receptor to a receptor tyrosine kinase-like receptor (See e.g., Zhang, L., H. H. Loh, and P. Y. Law, A novel noncanonical signaling pathway for the mu-opioid receptor. Mol Pharmacol, 2013. 84(6): p. 844-53). Additionally, Src has been reported to participate in delta opioid receptor activation of ERK (See e.g., Audet, N., et al., Internalization and Src activity regulate the time course of ERK activation by delta opioid receptor ligands. J Biol Chem, 2005. 280(9): p. 7808-16). Src activation by chronic opioids and consequent phosphorylation of multiple signaling molecules could concomitantly activate parallel tolerant mechanisms, all of which might be eliminated by Src inhibition. Without wishing to be bound by the present disclosure, this could account for the ability of PP2 to interrupt opioid tolerance, however it would not explain the ability of CSD to do the same, as was currently found. The most parsimonious explanation for our finding that PP2 and CSD have concordant effects on opioid antiallodynic tolerance would be that PP2 and CSD acted at sequential steps in a common mechanism, PP2 inhibiting Src phosphorylation of Cav1, preventing the altered scaffolding that is critical to antiallodynic morphine tolerance, CSD eliminating Cav1 scaffolding by competing with Cav1 binding to signaling molecules.

Many cellular adaptations to chronic morphine have been described in cells maintained in culture and laboratory animal models. It remains unclear whether the lack of clinical utility of the most, if not all, of the previously intimated opioid tolerance mitigating therapies resulted from failure of the test or failure to test. Regardless, theoretically, there is a much greater likelihood for clinical success of developing opioid anti-tolerance pharmacotherapies that disrupt Cav1 scaffolding functionality in the presence of chronic opioids since doing so has the potential to concomitantly interrupt multiple analgesic/antiallodynic tolerance mechanisms.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed is:

1. A method for preventing and/or reducing opioid analgesic tolerance in a subject treated for chronic pain comprising:
administering, to the subject, an effective amount of a caveolin-1 scaffolding domain (CSD) polypeptide,
wherein the subject has chronic pain and is administered chronic opioid analgesic therapy prior to or concurrently with the administration of the CSD polypeptide, and
wherein said administration of the CSD polypeptide prevents or reduces analgesic tolerance in the subject.

2. The method of claim 1, wherein said subject is administered the chronic opioid analgesic therapy concurrently with the CSD polypeptide, for at least one day prior to the administration of the CSD polypeptide, for at least six days prior to the administration of the CSD polypeptide, or the subject has not been administered opioid analgesic therapy prior to the administration of the CSD polypeptide.

3. The method of any of claim 1 or 2, wherein said analgesic is an opioid selected from the group consisting of morphine, fentanyl, hydrocodone, hydromorphone, meperidine, oxycontin, oxycodone, and combinations thereof.

4. The method of any of claims 1 to 3, wherein the chronic opioid analgesic therapy is administered either systemically, such as subcutaneously, or to the central nervous system such as intrathecally.

5. The method of any of claims 1 to 4, wherein the CSD polypeptide is administered by systemic or spinal injection.

6. The method of any of claims 1 to 5, wherein the CSD polypeptide comprises an amino acid sequence consisting of SEQ ID NO:1 or SEQ ID NO:2, or an amino acid sequence having at least 90% sequence identity to SEQ ID NO:1 or SEQ ID NO:2.

7. A method for preventing and/or reducing opioid analgesic tolerance in a subject treated for chronic pain comprising:
administering to the subject an effective amount of a non-receptor tyrosine kinase (c-Src) inhibitor, wherein the subject has chronic pain and is administered chronic opioid analgesic therapy prior to or concurrently with the administration of the c-Src inhibitor, and
wherein said administration of the c-Src inhibitor prevents or reduces opioid analgesic tolerance in the subject.

8. The method of claim 7, wherein said subject is administered the chronic opioid analgesic therapy concurrently with the c-Src, for at least one day prior to the administration of the c-Src inhibitor, for at least six days prior to the administration of the c-Src inhibitor, or the subject has not been administered opioid analgesic therapy prior to the administration of the c-Src inhibitor.

9. The method of claim 7 or 8, wherein said analgesic is an opioid selected from the group consisting of morphine, fentanyl, hydrocodone, hydromorphone, meperidine, oxycontin, oxycodone, and combinations thereof.

10. The method of any of claims 7 to 9, wherein the c-Src inhibitor comprises 4-Amino-5-(4-chlorophenyl)-7-(dimethylethyl)pyrazolo[3,4-d]pyrimidine (PP2).

11. A method for preventing and/or reducing opioid analgesic tolerance in a subject treated for chronic pain comprising:
administering to the subject an effective amount of: a caveolin-1 scaffolding domain (CSD) polypeptide and a non-receptor tyrosine kinase (c-Src) inhibitor, wherein the subject has chronic pain and is administered chronic opioid analgesic therapy prior to or concurrently with the administration of the CSD polypeptide and the c-Src inhibitor, and wherein said administration of the CSD polypeptide and the c-Src inhibitor prevents or reduces opioid analgesic tolerance in the subject.

12. The method of claim 11, wherein said subject is administered the chronic opioid analgesic therapy concurrently with the CSD polypeptide and the c-Src inhibitor, for at least one day prior to the administration of the CSD polypeptide and the c-Src inhibitor, for at least six days prior to the administration of the CSD polypeptide and the c-Src inhibitor, or the subject has not been administered opioid analgesic therapy prior to the administration of the CSD polypeptide and the c-Src inhibitor.

13. The method of claim 11 or 12, wherein said analgesic is an opioid selected from the group consisting of morphine, fentanyl, hydrocodone, hydromorphone, meperidine, oxycontin and oxycodone, and combinations thereof.

14. The method of any of claims 11 to 13, wherein the chronic opioid analgesic therapy is administered systemically or spinally, and wherein the CSD polypeptide and the c-Src inhibitor are administered by intrathecal injection.

15. A method of treating, ameliorating, or preventing one or more symptoms of pain in a subject, comprising:
administering a therapeutically effective amount of an opioid to a subject; and
administering a therapeutically effective amount of a polypeptide having at least 90% sequence identity to SEQ ID NO: 1, or SEQ ID NO: 2, a c-Src inhibitor, or a polypeptide having at least 90% sequence identity to SEQ ID NO: 1 or SEQ ID NO: 2, and a c-Src inhibitor to a subject in need thereof.


SEQUENCE LISTING (3 sequences)

SEQ ID NO: 1 - protein, 36 aa, Artificial Sequence
  Synthetic sequence
RQIKIWFQNRRMKWKKDGIWKASFTTFTVTKYWFYR

SEQ ID NO: 2 - protein, 20 aa, Artificial Sequence
  Synthetic sequence
DGIWKASFTTFTVTKYWFYR

SEQ ID NO: 3 - protein, 36 aa, Artificial Sequence
  Synthetic Sequence
RQIKIWFQNRRMKWKKWGIDKASFTTFTVTKYWFRY